(19) United States
(12) Patent Application Publication
(10) Pub. No.: US 2025/0090514 A1
(43) Pub. Date: Mar. 20, 2025

(54) METHOD OF TREATING CANCER

(71) Applicant: Laekna Limited, Admiralty (HK)

(72) Inventors: Chris Lu, Lexington, MA (US); Ruipeng Zhang, Shanghai (CN); Yong Yue, Warren, NJ (US); Minhua Zhang, Shanghai (CN)

(21) Appl. No.: 18/968,073

(22) Filed: Dec. 4, 2024

Related U.S. Application Data
(63) Continuation of application No. 16/987,924, filed on Aug. 7, 2020.

(30) Foreign Application Priority Data
Aug. 8, 2019 (WO) PCT/CN2019/099754

(51) Int. Cl. A61K 31/444; A61K 31/4155; A61K 31/573; A61P 35/00

(52) U.S. Cl. CPC A61K 31/444; A61K 31/4155; A61K 31/573; A61P 35/00

(57) ABSTRACT

The present application provides a method of treating castrate resistant prostate cancer in a patient, comprising administering to the patient a combination of afuresertib and CFG920, wherein the castrate resistant prostate cancer is also resistant to one or more standard of care treatments.

### CLAIM OF PRIORITY

This application is a continuation application of U.S. application Ser. No. 16/987,924 filed Aug. 7, 2020, which claims priority and the benefit of International Application Serial No. PCT/CN2019/099754, filed on Aug. 8, 2019. The entire contents of the foregoing are incorporated herein by reference.

### FIELD OF INVENTION

The present disclosure relates to a method of treating cancer and to combinations useful in such treatment.

### BACKGROUND

Effective treatment of hyperproliferative disorders including cancer is a continuing goal in the oncology field. Generally, cancer results from the deregulation of the normal processes that control cell division, differentiation and apoptotic cell death and is characterized by the proliferation of malignant cells, which have the potential for unlimited growth, local expansion and systemic metastasis. Deregulation of normal processes includes abnormalities in signal transduction pathways, and/or abnormalities in the regulation of gene transcription, and/or responses to factors (e.g., growth factors) which differ from those found in normal cells.

Prostate cancer is characterized by dependence on the androgen signaling pathway. Certain specific genetic alterations in the androgen receptor could activate the androgen signaling pathway and promote prostate cancer cell growth. The primary mode of treatment for metastatic prostate cancer has historically focused on targeting androgen-androgen receptor signaling by either decreasing the amount of ligand (androgens) available for binding to the androgen receptor or blocking androgen receptor binding with its ligand, the two major kinds of anti-prostate cancer medicines used in the clinic.

A first kind of anti-prostate cancer medicine is androgen antagonists, also known as antiandrogens. Antiandrogens alter the androgen pathway by blocking the receptor, competing for binding sites on the cell's surface or affecting androgen production. The most common antiandrogens are androgen receptor antagonists, which act on the target cell level and competitively bind to androgen receptors. By competing with circulating androgens for binding sites on prostate cell receptors, antiandrogens promote apoptosis and inhibit prostate cancer growth.

A second kind of anti-prostate cancer medicine is inhibitors of androgen synthesis enzyme. Cytochrome P450 17A1, also known as 17 α-hydroxylase/C17,20 lyase (CYP17A1), is a key enzyme in the pathway that produces progestin, mineralocorticoid, glucocorticoid, androgen and estrogen. Inhibition of CYP17A1 provides an effective therapeutic tool in targeting the androgen-receptor (AR) signaling pathway; however, when this pathway is activated at the post-receptor ligand binding level or through non-hormonally mediated mechanisms, CYP17A1 inhibitors may not suffice. Without intending to be limited to any particular theory, this implies that additional factors may be involved in prostate cancer cell growth and may be related to the development of drug-resistance following the use of CYP17A1 inhibitors. (Rini, B. I., and Small, E. J., Hormone-refractory prostate cancer. Cuf. Treat. Options Oncol. 2002; 3:437; Singh, P., Yam, M., Russell, P. J., and Khatri, A., Molecular and traditional chemotherapy: a united front against prostate cancer. Cancer Lett. 2010;293:1).

Many prostate cancers are also characterized by constitutive or otherwise abnormal activation of the phosphoinositide 3-kinase (PI3K) signaling pathway. The PI3K pathway is among the most commonly activated in human cancer and the importance in carcinogenesis is well established (Samuels Y and Ericson K. Oncogenic PI3K and its role in cancer. Current Opinion in Oncology, 2006; 18:77-82). Initiation of signaling begins with the phosphorylation of phosphatidylinositol-4, 5-bisphosphate (PIP2) to produce phosphatidylinositol-3, 4, 5-P3 (PIP3). PIP3 is a critical second messenger, which recruits proteins that contain pleckstrin homology domains to the cell membrane where they are activated. The most studied of these proteins is protein kinase B (AKT) which promotes cell survival, growth, and proliferation. It has been shown that in many cases the mechanism of activation of PI3K signaling in prostate cancers is functional deficiencies of the tumor suppressor protein phosphatase and tensin (PTEN).

Androgen deprivation therapy remains the standard of care for treatment of advanced prostate cancer. Despite an initial favorable response, almost all patients invariably progress to a more aggressive, castrate-resistant phenotype. Evidence indicates that the development of castrate-resistant prostate cancer is causally related to continue signaling of the androgen receptor. Prostate cancer that has progressed despite castrate levels of androgens (<50 ng/mL) is termed castrate resistant prostate cancer (CRPC). Abiraterone and enzalutamide are the approved drugs for the treatment of metastatic castrate resistant prostate cancer (mCRPC) after chemotherapy. Abiraterone is an irreversible inhibitor of CYP17, a key enzyme for both adrenal and intra-tumoral androgen synthesis, whereas enzalutamide is an androgen receptor antagonist. Both abiraterone and enzalutamide reduce patient's androgen signal levels to block prostate cancer growth. However, most patients responding to abiraterone and enzalutamide eventually develop resistance. The currently available treatments for such cancers are limited to rotating abiraterone, enzalutamide and chemotherapy with a median PFS around 2.8 to 4.0 months in mCRPC patients who failed prior treatments (de Bono, et al., Eur Urol. 2018;74(1):37-45.; Caffo, et al., Eur Urol. 2015;68(1):147-53.). However, a cross-resistance between the taxanes (docetaxel and cabazitaxel) and AR targeting agents abiraterone and enzalutamide was reported recently in the literature, which makes clinical management with the current treatment strategy for treatment-resistant mCRPC patients even more challenging (van Soest, et al., Eur J Cancer. 2013;49(18):3821-30.; Shiota, et al., Cancer Sci. 2018;109(10):3224-34. doi: 10.1111/cas.13751.). Therefore, there is a large unmet medical need to develop new therapies for treatment-resistant mCRPC patients.

### SUMMARY

The present application provides, inter alia, a method of treating castrate resistant prostate cancer in a patient, wherein the patient is resistant to one or more prostate cancer treatments, comprising administering to the patient:
(i) N-{(1S)-2-amino-1-[(3-fluorophenyl)methyl]ethyl}-5-chloro-4-(4-chloro-1-methyl-1H-pyrazol-5-yl)-2-thiophenecarboxamide (afuresertib), or a pharmaceutically acceptable salt thereof;
(ii) 1-(2-chloro-pyridin-4-yl)-3-(4-methyl-pyridin-3-yl)-imidazolidin-2-one (CFG920), or a pharmaceutically acceptable salt thereof; and
(iii) optionally a corticosteroid, such as prednisone.

The details of one or more embodiments of the invention are set forth in the description below. Other features, objects, and advantages of the invention will be apparent from the description and from the claims.

### DESCRIPTION OF DRAWINGS

FIGS. 1A-1D are graphs showing the anti-tumor efficacy of CFG920 and afuresertib in the treatment of MDX191210 in a MiniPDX mouse model. The values are presented as Mean±SEM. FIG. 1A shows relative luminescence unit (RLU) values for each study group as determined in a Cell Titer-Glo (CTG) assay. FIG. 1B shows relative tumor proliferation values (%) of each study group. FIG. 1C shows changes in body weight in grams for each study group. FIG. 1D shows relative changes in body weight (RCBW, reported as percentage change from day 0) in each study group.

FIGS. 2A-2C are graphs showing the anti-tumor efficacy of CFG920 and afuresertib in a human patient. FIG. 2A shows PSA level changes of the patient before and after receiving study treatments (Cut-off day: Jul. 23, 2020). FIG. 2B shows testosterone level changes of the patient under the study treatments. FIG. 2C shows aldosterone level changes of the patient under the study treatments.

### DETAILED DESCRIPTION

CFG920 is a novel nonsteroidal, reversible, dual inhibitor of cytochrome 17A1 (CYP17A1) (an enzyme for testosterone synthesis) and CYP11B2 (aldosterone synthase). A first-in-human Phase I/II study has been conducted in metastatic castration-resistant prostate cancer (mCRPC) patients by Novartis Pharmaceuticals. (See ClinicalTrials.gov Identifier: NCT01647789). This study evaluated safety, recommended Phase II dose (RP2D), pharmacokinetic data (PK), and pharmacodynamics data (PD) and achieved proof of concept in mCRPC patients by demonstrating that the unconfirmed rate of ≥50% prostate-specific antigen (PSA) decline from baseline in this study was 28% (16 of 57; 95% confidence interval [CI]: 17 to 42) and 26% (32 of 124; 95% CI: 18 to 34) for patients with previous chemotherapy and chemotherapy-naive patients, respectively. For the mCRPC patients, the most commonly used medicines have been changed with the approval of enzalutamide and abiraterone acetate (Beer, et al., J Clin Oncol. 2014;32(Suppl_4), abs LBA1.;Scher, et al., N Engl J Med. 2012;367(13):1187-97.; Shore, et al., Lancet Oncol. 2016;17(2):153-63.; de Bono, et al., N Engl J Med. 2011;364:1995-2005.; Ryan, et al., N Engl J Med. 2013;368(2):138-48.). However, a more modest response to abiraterone following progression on docetaxel and enzalutamide was observed in a limited number of patients with mCRPC, including only 8% of patients achieving ≥50% decline in PSA on subsequent abiraterone. Median time to progression (PSA, objective or symptomatic) following abiraterone treatment was only 15.4 weeks (95% CI 10.7 to 20.2) (Loriot, et al., Ann Oncol. 2013;24(7):1807-12.; Noonan, et al., Ann Oncol. 2013;24(7):1802-7.).

Without intending to be limited to any particular theory, one of the potential reasons for the low PSA response rates and short progression free survival (PFS) in mCRPC patients following enzalutamide and abiraterone plus prednisone treatments is that other factors besides androgen-signal pathways also contribute to prostate cancer cell growth and differentiation. The phosphatidylinositol 3-kinase (PI3K)/AKT pathway has been implicated in prostate carcinogenesis and castration resistance, although the precise function of the PI3K/AKT pathway remains to be fully elucidated (Chen, et al., Front Biosci (Landmark Ed). 2016;21:1084-91.). Activated AKT translocates to the cytoplasm and nucleus and activates downstream targets involved in survival (Wegie, et al., Int J Cancer. 2008;122(7):1521 9.; Lee, et al., Mol Cancer. 2004;3:31. doi: 10.1186/1476-4598-3-31.), proliferation (Gao, et al., Biochem Biophys Res Commun. 2003;310(4):1124 32.) and apoptosis (Kim, et al. Phytother Res. 2014;28(3):423-31.) of prostate cancer cells, in addition to migration and invasion (Vo, et al., Endocrinology. 2013; 154 (5): 1768-79.). The tumor suppressor phosphatase and tensin homolog (PTEN) deletion on chromosome 10 is recognized as a major inhibitor of PI3K and AKT (Sansal, et al. J Clin Oncol. 2004;22(14):2954-63.; Carnero, et al., Curr Cancer Drug Targets. 2008;8(3):187-98.) pathways and is frequently lost in human tumors. Prostate cancer is one of the cancers most commonly affected by PTEN abnormalities (Sulis, et al., Trends Cell Biol. 2003;13(9):478 83.). The biomarker for PI3K/AKT pathway activation and PTEN status was shown to be the insulin growth factor-binding protein 2 in prostate cancer (Mehrian-Shai, et al., Proc Natl Acad Sci USA. 2007;104(13):5563-8.). Bortezomib has been studied for use in in vitro prostate cancer treatment, wherein it was found to dephosphorylate phospho-AKT, leading to the suppression of PI3K/AKT/mTOR signals, resulting in induction of growth arrest and apoptosis in prostate cancer cells (Befani, et al., J Mol Med (Berl). 2012;90(1):45-54.). In addition, inactivation of PTEN by deletion or mutation is identified in approximately 16-20% of primary prostate tumor samples at radical prostatectomy and >50% of castration-resistant tumors (Hamid, et al. Eur Urol. 2019July;76(1):89-97; Jamaspishvili, et al. Nat Rev Urol. 2018;15(4):222-234.). PTEN loss is present in >60% of the mCRPC patients who failed prior docetaxel treatment, thus implying that PTEN loss-caused AKT pathway activation plays a critical role in the progression of mCRPC after failure of prior standard treatments (de Bono, et al. Annals of Oncology. 2016; 27 (Suppl_6):243-265.).

A clinical study reported that about more than 70% of CRPC patients respond to first-line treatment with abiraterone or enzalutamide initially (de Bono, et al., N Engl J Med. 2011;364:1995-2005). However, a subsequent PSA increase, or tumor progression occurred in nearly all of the responders around 15 months (de Bono, et al., Eur Urol. 2018;74(1):37-45). The increasing percentage of patients with PTEN loss with the progress of the CRPC causes the activation of the PI3K/AKT pathways that may play a critical role as a major mechanisms of abiraterone-and/or enzalutamide-resistance.

The present application provides a method of treating castrate resistant prostate cancer in a patient, comprising administering to the patient:
(i) N-{(1S)-2-amino-1-[(3-fluorophenyl) methyl]ethyl}-5-chloro-4-(4-chloro-1-methyl-1H-pyrazol-5-yl)-2-thiophenecarboxamide (afuresertib), or a pharmaceutically acceptable salt thereof;
(ii) 1-(2-chloro-pyridin-4-yl)-3-(4-methyl-pyridin-3-yl)-imidazolidin-2-one (CFG920), or a pharmaceutically acceptable salt thereof; and
(iii) optionally a corticosteroid;
wherein the patient is resistant to one or more prostate cancer treatments. Afuresertib has the following chemical structure:

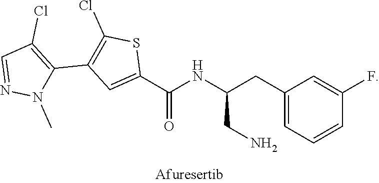 [SMILES: Cn1ncc(Cl)c1-c1cc(C(=O)N[C@H](CN)Cc2cccc(F)c2)sc1Cl]

CFG920 has the following chemical structure:

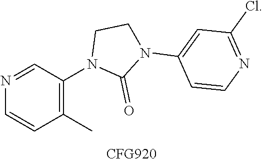 [SMILES: Cc1ccncc1N1CCN(c2ccnc(Cl)c2)C1=O]

In some embodiments, the patient is resistant to one or more prior prostate cancer treatments. The prostate cancer treatments can include treatments with one or more anti-androgen agent, a chemotherapeutic agent, or a combination thereof. In some embodiments, the patient is resistant to one or more standard of care prostate cancer treatments, which can comprise one or more of anti-androgen agent, chemotherapeutic agent, or a combination thereof. In some embodiments, the patient is resistant to treatments comprising one or more of an anti-androgen agent, a chemotherapeutic agent, or a combination thereof. In some embodiments, the patient is resistant to at least two anti-androgen agents. In some embodiments, the patient is resistant to at least one anti-androgen agent and at least one chemotherapeutic agent. In some embodiments, the anti-androgen agent comprises one or more of abiraterone, enzalutamide, apalutamide, and darolutamide or a pharmaceutically acceptable salt or prodrug thereof. In some embodiments, the chemotherapeutic agent comprises one or more of docetaxel and cabazitaxel, or a pharmaceutically acceptable salt or prodrug thereof. In some embodiments, the patient is suffering from castrate resistant prostate cancer and has phosphatase and tensin homolog (PTEN) loss. In some embodiments, the patient is determined to be resistant based on prior treatment results. For example, the patient is determined to be resistant through identification of one or more biomarkers that have been associated with resistance with one or more treatment therapies. In some embodiments, the patient is determined to be resistant through genomic analysis. In some embodiments, the patient is determined to be resistant through in vitro testing of biopsied tissue samples.

In some embodiments, provided herein is a method of treating castrate resistant prostate cancer in a patient, comprising administering to the patient afuresertib, or a pharmaceutically acceptable salt thereof; CFG920, or a pharmaceutically acceptable salt thereof; and optionally a corticosteroid.

In some embodiments, the castrate resistant prostate cancer is metastatic castrate resistant prostate cancer (mCRPC).

Abiraterone and pharmaceutically acceptable salts thereof are 17α-Hydroxylase/C_{17,20}-lyase inhibitors. Abiraterone acetate (CAS Registry NO. 154229-18-2), is a compound known by the chemical name (3S, 10R, 13S)-10,13-dimethyl-17-pyridin-3-yl-2,3,4,7,8,9,11,12, 14, 15-decahydro-1H-cyclopenta[a]phenanthren-3-yl]acetate) having the formula shown below. Abiraterone acetate is commercially available as ZYTIGA® from Janssen Biotech, Inc. and disclosed in PCT International Application WO 93/20097, the contents of which are incorporated herein by reference. Abiraterone acetate is converted in vivo to abiraterone, an androgen biosynthesis inhibitor, that inhibits CYP17 (17α-hydroxylase/C_{17,20}-lyase).

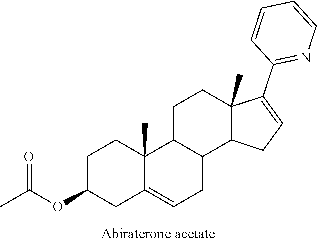 [SMILES: CC(=O)O[C@H]1CC[C@@]2(C)C(=CCC3C2CC[C@]2(C)C(c4ccccn4)=CCC32)C1]

Enzalutamide and pharmaceutically acceptable salts thereof are androgen receptor inhibitors. Enzalutamide is commercially available as XTANDI® from Pfizer Inc./Astellas Pharma US, Inc. IUPAC name 4-(3-(4-cyano-3-(trifluoromethyl) phenyl)-5,5-dimethyl-4-oxo-2-thioxoimidazolidin-1-y1)-2-fluoro-N-methylbenzamide. CAS No. 915087-33-1. Enzalutamide was first described in US Patent Application Publication US2007/0004753A1, the contents of which are incorporated herein by reference.

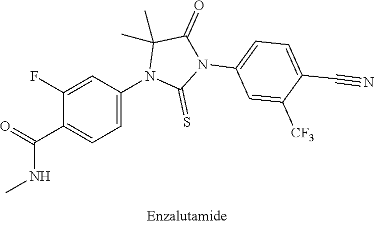 [SMILES: CNC(=O)c1ccc(N2C(=S)N(c3ccc(C#N)c(C(F)(F)F)c3)C(=O)C2(C)C)cc1F]

Apalutamide and pharmaceutically acceptable salts thereof are androgen receptor inhibitors. Apalutamide is commercially available as ERLEADA® from Janssen Biotech, Inc. IUPAC name 4-[7-[6-cyano-5-(trifluoromethyl)pyridin-3-yl]-8-oxo-6-sulfanylidene-5,7-diazaspiro[3.4]octan-5-yl]-2-fluoro-N-methylbenzamide. CAS No. 956104-40-8. Apalutamide was first described in PCT Patent Application Publication WO 2007/126765, the contents of which are incorporated herein by reference.

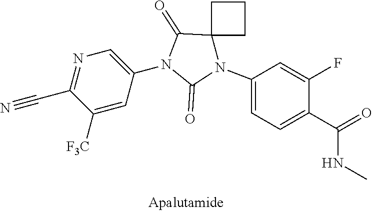 [SMILES: CNC(=O)c1ccc(N2C(=O)N(c3cnc(C#N)c(C(F)(F)F)c3)C(=O)C23CCC3)cc1F]

Darolutamide and pharmaceutically acceptable salts thereof are androgen receptor antagonists. Darolutamide is under development by Orion Oyj and Bayer HealthCare for the treatment of castration-resistant prostate cancer under developmental names ODM-201 and BAY-1841788. IUPAC name N-((S)-1-(3-(3-Chloro-4-cyanophenyl)-1H-pyrazol-1-yl)propan-2-yl)-5-(1-hydroxyethyl)-1H-pyrazole-3-carboxamide. CAS No. 1297538-32-9. Darolutamide was first described in PCT Patent Application Publication WO 2011/051540, the contents of which are incorporated herein by reference.

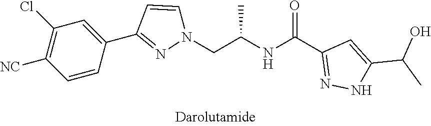 [SMILES: CC(O)c1cc(C(=O)N[C@@H](C)Cn2ccc(-c3ccc(C#N)c(Cl)c3)n2)n[nH]1]

Docetaxel and pharmaceutically acceptable salts thereof are taxane based chemotherapeutic agents commonly used in the treatment of various cancers, including breast, lung, prostate, gastric, head and neck, and ovarian cancer. Docetaxel is commercially available as TAXOTERE® from Sanofi-Aventis and was first disclosed in French patent application publication FR2601675A1. IUPAC name [(1S,2S,3R,4S,7R,9S,10S,12R,15S)-4-acetyloxy-1,9,12-trihydroxy-15-[(2R,3S)-2- hydroxy-3-[(2-methylpropan-2-yl)oxycarbonylamino]-3-phenylpropanoyl]oxy-10,14,17,17- tetramethyl-11-oxo-6-oxatetracyclo[11.3.1.0^{3,10}.0^{4,7}]heptadec-13-en-2-yl] benzoate. CAS No. 114977-28-5.

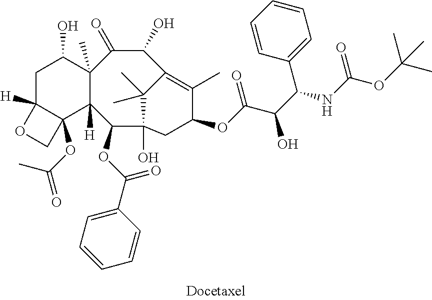 [SMILES: CC(=O)O[C@@]12CO[C@@H]1C[C@H](O)[C@@]1(C)C(=O)[C@H](O)C3=C(C)[C@@H](OC(=O)[C@H](O)[C@@H](NC(=O)OC(C)(C)C)c4ccccc4)C[C@@](O)([C@@H](OC(=O)c4ccccc4)[C@H]21)C3(C)C]

Cabazitaxel and pharmaceutically acceptable salts thereof are taxane based chemotherapeutic agents. Cabazitaxel is commercially available as JEVTANA® from Sanofi-Aventis and was first disclosed in PCT application publication WO 96/30355. IUPAC name (2α,5β, 7β, 10β, 13α)-4- acetoxy-13-({(2R,3S)-3-[(tertbutoxycarbonyl)amino]-2-hydroxy-3-phenylpropanoyl}oxy)-1- hydroxy-7,10-dimethoxy-9-oxo-5,20-epoxytax-11-en-2-yl benzoate. CAS No. 183133-96-2.

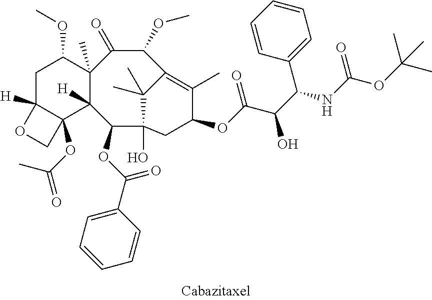

Methods of making afuresertib are described in U.S. Pat. Nos. 8,410,158 and 8,609,711. In some embodiments, afuresertib is in the form of a hydrochloride salt. In some embodiments, afuresertib is in the form of a hydrochloride salt having e.g., a 1:1 stoichiometric ratio of N-{(1S)-2- amino-1-[(3-fluorophenyl)methyl]ethyl}-5-chloro-4-(4-chloro-1-methyl-1H-pyrazol-5-yl)-2-thiophenecarboxamide to hydrochloric acid. In some embodiments, afuresertib is in the form of crystalline N-{(1S)-2-amino-1-[(3-fluorophenyl)methyl]ethyl}-5-chloro-4-(4-chloro-1-methyl-1H-pyrazol-5-yl)-2-thiophenecarboxamide hydrochloride. In some embodiments, the crystalline hydrochloride salt has one or more characteristic diffraction peaks in terms of 2-theta (±0.3°) selected from 7.2°, 14.4°, 17.9°, 18.5°, 20.8°, 21.5°, 22.4°, 22.9°, 23.7°, 24.5°, 24.7°, 25.1°, 25.7°, 27.3°, 28.2°, 28.8°, 30.4°, 32.4°, 32.7°, 35.2°, 36.1°, 40.0°, 41.3°, and 41.7°, as measured in an Powder X-Ray Diffractogram using Cu Kα radiation. In some embodiments, the crystalline hydrochloride salt has a DSC thermogram having an endothermic peak at about 220° C. Methods of making crystalline afuresertib and salts thereof are described in U.S. Pat. No. 8,609,711.

Methods of making CFG920 are described in U.S. Pat. No. RE45,173. In some embodiments, the CFG920 is in the form of a free base. In some embodiments, the CFG920 is in a crystalline form. In some embodiments, the CFG920 is the free base, in an anhydrous crystalline form. In some embodiments, the anhydrous crystalline free base has one or more characteristic diffraction peaks in terms of 2-theta (±0.3°) selected from 12.7°, 13.5°, 15.7°, 17.2°, 18.7°, 19.1°, 20.0°, 20.6°, 22.2°, 24.1°, 25.6°, 26.1°, 26.5°, 27.1°, and 27.8°, as measured in an Powder X-Ray Diffractogram using Cu Ka radiation. In some embodiments, the anhydrous crystalline free base has a DSC thermogram having an endothermic peak at about 175° C.

In some embodiments, afuresertib, or a pharmaceutically acceptable salt thereof, is administered to the patient in a total daily dosage of:
(i) from about 1 mg to about 1,000 mg; or
(ii) from about 1 mg to about 500 mg; or
(iii) from about 10 mg to about 500 mg; or
(iv) from about 20 mg to about 400 mg; or
(v) from about 30 mg to about 300 mg; or
(vi) from about 40 mg to about 250 mg; or
(vii) from about 50 mg to about 200 mg; or
(viii) from about 75 mg to about 150 mg; or
(ix) from about 75 mg to about 100 mg;
on a free base basis.

In some embodiments, the afuresertib, or a pharmaceutically acceptable salt thereof, is administered to the patient in a total daily dosage of about 20 mg, about 25 mg, about 30 mg, about 40 mg, about 50 mg, about 60 mg, about 70 mg, about 75 mg, about 80 mg, about 90 mg, about 100 mg, about 110 mg, about 120 mg, about 125 mg, about 130 mg, about 140 mg, or about 150 mg, on a free base basis. In some embodiments, the afuresertib, or a pharmaceutically acceptable salt thereof, is administered to the patient in a total daily dosage of about 75 mg, about 100 mg, about 125 mg or about 150 mg, on a free base basis.

In some embodiments, the afuresertib, or a pharmaceutically acceptable salt thereof, is administered to the patient once daily (QD). In some embodiments, afuresertib, or a pharmaceutically acceptable salt thereof, is administered to the patient in a dosage of from about 75 mg to about 150 mg, on a free base basis, once per day. In some embodiments, afuresertib, or a pharmaceutically acceptable salt thereof, is administered to the patient in a dosage of about 10 mg, about 20 mg, about 25 mg, about 30 mg, about 40 mg, about 50 mg, about 60 mg, about 70 mg, about 75 mg, about 80 mg, about 90 mg, about 100 mg, about 110 mg, about 120 mg, about 125 mg, about 130 mg, about 140 mg, or about 150 mg, on a free base basis, once per day. In some embodiments, afuresertib, or a pharmaceutically acceptable salt thereof, is administered to the patient in a dosage of about 75 mg, about 100 mg, about 125 mg or about 150 mg, on a free base basis, once per day.

In some embodiments, CFG920, or a pharmaceutically acceptable salt thereof, is administered to the patient in a total daily dosage of:
(i) from about 1 mg to about 1,000 mg; or
(ii) from about 1 mg to about 750 mg; or
(iii) from about 10 mg to about 750 mg; or
(iv) from about 20 mg to about 500 mg; or
(v) from about 30 mg to about 400 mg; or
(vi) from about 40 mg to about 300 mg; or
(vii) from about 50 mg to about 300 mg; or
(viii) from about 75 mg to about 300 mg; or
(ix) from about 100 mg to about 250 mg; or
(x) from about 125 mg to about 200 mg; or
(xi) from about 150 mg to about 200 mg; or
(xii) from about 75 mg to about 200 mg;
on a free base basis.

In some embodiments, CFG920, or a pharmaceutically acceptable salt thereof, is administered to the patient in a total daily dosage of about 10 mg, about 20 mg, about 25 mg, about 30 mg, about 40 mg, about 50 mg, about 60 mg, about 70 mg, about 75 mg, about 80 mg, about 90 mg, about 100 mg, about 110 mg, about 120 mg, about 125 mg, about 130 mg, about 140 mg, about 150 mg, about 160 mg, about 170 mg, about 175 mg, about 180 mg, about 190 mg, about 200 mg, about 210 mg, about 220 mg, about 225 mg, about 230 mg, about 240 mg, about 250 mg, about 260 mg, about 270 mg, about 275 mg, about 280 mg, about 290 mg, of about 300 mg, on a free base basis. In some embodiments, CFG920, or a pharmaceutically acceptable salt thereof, is administered to the patient in a total daily dosage of about 150 mg or about 200 mg.

In some embodiments, CFG920, or a pharmaceutically acceptable salt thereof, is administered to the patient twice per day (BID). For example, a total daily dosage of 150 mg of CFG920 could be administered twice daily as 75 mg per dose. In some embodiments, CFG920, or a pharmaceutically acceptable salt thereof, is administered to the patient in a dosage of:
(i) from about 1 mg to about 1,000 mg per dose, twice per day (total daily dosage of about 2 mg to about 2,000 mg); or
(ii) from about 1 mg to about 500 mg per dose, twice per day (total daily dosage of about 2 mg to about 1,000 mg); or
(iii) from about 10 mg to about 500 mg per dose, on a free base basis, twice per day (total daily dosage of about 20 mg to about 1,000 mg); or
(iv) from about 20 mg to about 400 mg per dose, on a free base basis, twice per day (total daily dosage of about 40 mg to about 800 mg); or
(v) from about 30 mg to about 300 mg per dose, on a free base basis, twice per day (total daily dosage of about 60 mg to about 600 mg); or
(vi) from about 40 mg to about 250 mg per dose, on a free base basis, twice per day (total daily dosage of about 80 mg to about 500 mg); or
(vii) from about 50 mg to about 200 mg per dose, on a free base basis, twice per day (total daily dosage of about 100 mg to about 400 mg); or
(ix) from about 75 mg to about 125 mg per dose, on a free base basis, twice per day (total daily dosage of about 150 mg to about 250 mg); or
(x) from about 75 mg to about 100 mg per dose, on a free base basis, twice per day (total daily dosage of about 150 mg to about 200 mg);
on a free base basis.

In some embodiments, CFG920, or a pharmaceutically acceptable salt thereof, is administered to the patient in a twice daily dosage of about 10 mg, about 20 mg, about 25 mg, about 30 mg, about 40 mg, about 50 mg, about 60 mg, about 70 mg, about 75 mg, about 80 mg, about 90 mg, about 100 mg, about 110 mg, about 120 mg, about 125 mg, about 130 mg, about 140 mg, or about 150 mg per dose, on a free base basis. In some embodiments, CFG920, or a pharmaceutically acceptable salt thereof, is administered to the patient in a twice daily dosage of about 75 mg or about 125 mg per dose (total daily dosage of about 150 mg or about 250 mg). In some embodiments, CFG920, or a pharmaceutically acceptable salt thereof, is administered to the patient in a twice daily dosage of about 75 mg or about 100 mg per dose (total daily dosage of about 150 mg or about 200 mg).

In some embodiments, the corticosteroid is prednisone. In some embodiments, the prednisone is administered to the patient in a total daily dosage of about 10 mg. In some embodiments, the prednisone is administered to the patient twice per day (BID). In some embodiments, the prednisone is administered to the patient in a twice daily dosage of about 5 mg per dose.

In some embodiments, the patient is administered a twice daily dosage of about 50 mg per dose of CFG920 (total daily dosage of about 100 mg), or a pharmaceutically acceptable salt thereof, on a free base basis; and a once daily dosage of about 75 mg per dose of afuresertib, or a pharmaceutically acceptable salt thereof, on a free base basis.

In some embodiments, the patient is administered a twice daily dosage of about 50 mg per dose of CFG920 (total daily dosage of about 100 mg), or a pharmaceutically acceptable salt thereof, on a free base basis; and a once daily dosage of about 100 mg per dose of afuresertib, or a pharmaceutically acceptable salt thereof, on a free base basis.

In some embodiments, the patient is administered a twice daily dosage of about 50 mg per dose of CFG920 (total daily dosage of about 100 mg), or a pharmaceutically acceptable salt thereof, on a free base basis; and a once daily dosage of about 125 mg per dose of afuresertib, or a pharmaceutically acceptable salt thereof, on a free base basis.

In some embodiments, the patient is administered a twice daily dosage of about 75 mg per dose of CFG920 (total daily dosage of about 150 mg), or a pharmaceutically acceptable salt thereof, on a free base basis; and a once daily dosage of about 75 mg per dose of afuresertib, or a pharmaceutically acceptable salt thereof, on a free base basis.

In some embodiments, the patient is administered a twice daily dosage of about 75 mg per dose of CFG920 (total daily dosage of about 150 mg), or a pharmaceutically acceptable salt thereof, on a free base basis; and a once daily dosage of about 100 mg per dose of afuresertib, or a pharmaceutically acceptable salt thereof, on a free base basis.

In some embodiments, the patient is administered a twice daily dosage of about 75 mg per dose of CFG920 (total daily dosage of about 150 mg), or a pharmaceutically acceptable salt thereof, on a free base basis; and a once daily dosage of about 125 mg per dose of afuresertib, or a pharmaceutically acceptable salt thereof, on a free base basis.

In some embodiments, the patient is administered a twice daily dosage of about 100 mg per dose of CFG920 (total daily dosage of about 200 mg), or a pharmaceutically acceptable salt thereof, on a free base basis; and a once daily dosage of about 100 mg per dose of afuresertib, or a pharmaceutically acceptable salt thereof, on a free base basis.

In some embodiments, the patient is administered a twice daily dosage of about 100 mg per dose of CFG920 (total daily dosage of about 200 mg), or a pharmaceutically acceptable salt thereof, on a free base basis; and a once daily dosage of about 125 mg per dose of afuresertib, or a pharmaceutically acceptable salt thereof, on a free base basis.

In some embodiments, the patient is administered a twice daily dosage of about 100 mg per dose of CFG920 (total daily dosage of about 200 mg), or a pharmaceutically acceptable salt thereof, on a free base basis; and a once daily dosage of about 150 mg per dose of afuresertib, or a pharmaceutically acceptable salt thereof, on a free base basis.

In some embodiments, the patient is administered a twice daily dosage of about 125 mg per dose of CFG920 (total daily dosage of about 250 mg), or a pharmaceutically acceptable salt thereof, on a free base basis; and a once daily dosage of about 150 mg per dose of afuresertib, or a pharmaceutically acceptable salt thereof, on a free base basis.

In some embodiments, the patient is administered a twice daily dosage of about 50 mg per dose of CFG920 (total daily dosage of about 100 mg), or a pharmaceutically acceptable salt thereof, on a free base basis; a twice daily dosage of about 5 mg per dose of prednisone (total daily dosage of about 10 mg); and a once daily dosage of about 75 mg per dose of afuresertib, or a pharmaceutically acceptable salt thereof, on a free base basis.

In some embodiments, the patient is administered a twice daily dosage of about 50 mg per dose of CFG920 (total daily dosage of about 100 mg), or a pharmaceutically acceptable salt thereof, on a free base basis; a twice daily dosage of about 5 mg per dose of prednisone (total daily dosage of about 10 mg); and a once daily dosage of about 100 mg per dose of afuresertib, or a pharmaceutically acceptable salt thereof, on a free base basis.

In some embodiments, the patient is administered a twice daily dosage of about 50 mg per dose of CFG920 (total daily dosage of about 100 mg), or a pharmaceutically acceptable salt thereof, on a free base basis; a twice daily dosage of about 5 mg per dose of prednisone (total daily dosage of about 10 mg); and a once daily dosage of about 125 mg per dose of afuresertib, or a pharmaceutically acceptable salt thereof, on a free base basis.

In some embodiments, the patient is administered a twice daily dosage of about 75 mg per dose of CFG920 (total daily dosage of about 150 mg), or a pharmaceutically acceptable salt thereof, on a free base basis; a twice daily dosage of about 5 mg per dose of prednisone (total daily dosage of about 10 mg); and a once daily dosage of about 75 mg per dose of afuresertib, or a pharmaceutically acceptable salt thereof, on a free base basis.

In some embodiments, the patient is administered a twice daily dosage of about 75 mg per dose of CFG920 (total daily dosage of about 150 mg), or a pharmaceutically acceptable salt thereof, on a free base basis; a twice daily dosage of about 5 mg per dose of prednisone (total daily dosage of about 10 mg); and a once daily dosage of about 100 mg per dose of afuresertib, or a pharmaceutically acceptable salt thereof, on a free base basis.

In some embodiments, the patient is administered a twice daily dosage of about 75 mg per dose of CFG920 (total daily dosage of about 150 mg), or a pharmaceutically acceptable salt thereof, on a free base basis; a twice daily dosage of about 5 mg per dose of prednisone (total daily dosage of about 10 mg); and a once daily dosage of about 125 mg per dose of afuresertib, or a pharmaceutically acceptable salt thereof, on a free base basis.

In some embodiments, the patient is administered a twice daily dosage of about 100 mg per dose of CFG920 (total daily dosage of about 200 mg), or a pharmaceutically acceptable salt thereof, on a free base basis; a twice daily dosage of about 5 mg per dose of prednisone (total daily dosage of about 10 mg); and a once daily dosage of about 100 mg per dose of afuresertib, or a pharmaceutically acceptable salt thereof, on a free base basis.

In some embodiments, the patient is administered a twice daily dosage of about 100 mg per dose of CFG920 (total daily dosage of about 200 mg), or a pharmaceutically acceptable salt thereof, on a free base basis; a twice daily dosage of about 5 mg per dose of prednisone (total daily dosage of about 10 mg); and a once daily dosage of about 125 mg per dose of afuresertib, or a pharmaceutically acceptable salt thereof, on a free base basis.

In some embodiments, the patient is administered a twice daily dosage of about 100 mg per dose of CFG920 (total daily dosage of about 200 mg), or a pharmaceutically acceptable salt thereof, on a free base basis; a twice daily dosage of about 5 mg per dose of prednisone (total daily dosage of about 10 mg); and a once daily dosage of about 150 mg per dose of afuresertib, or a pharmaceutically acceptable salt thereof, on a free base basis.

In some embodiments, the patient is administered a twice daily dosage of about 125 mg per dose of CFG920 (total daily dosage of about 250 mg), or a pharmaceutically acceptable salt thereof, on a free base basis; a twice daily dosage of about 5 mg per dose of prednisone (total daily dosage of about 10 mg); and a once daily dosage of about 150 mg per dose of afuresertib, or a pharmaceutically acceptable salt thereof, on a free base basis.

In some embodiments, afuresertib, or a pharmaceutically acceptable salt thereof, and CFG920, or a pharmaceutically acceptable salt thereof, are administered simultaneously. In some embodiments, afuresertib, or a pharmaceutically acceptable salt thereof, and CFG920, or a pharmaceutically acceptable salt thereof, are administered sequentially. In some embodiments, the corticosteroid and CFG920, or a pharmaceutically acceptable salt thereof, are administered simultaneously. In some embodiments, the corticosteroid and CFG920, or a pharmaceutically acceptable salt thereof, are co-formulated, whereby the corticosteroid and CFG920 are formulated together as part of a single pharmaceutical composition. In some embodiments, afuresertib, or a pharmaceutically acceptable salt thereof, is administered once per day and CFG920, or a pharmaceutically acceptable salt thereof, is administered twice per day, wherein afuresertib, or a pharmaceutically acceptable salt thereof, is administered simultaneously with one of the dosages of

CFG920, or a pharmaceutically acceptable salt thereof.

In some embodiments, the patient is administered a treatment regimen disclosed herein for a time period of up to several months. In some embodiments, the patient is administered a treatment regimen disclosed herein until the prostate cancer has progressed. In some embodiments, the patient is administered a treatment regimen disclosed herein until adverse effects are no longer tolerated. In some embodiments, the patient is administered a treatment regimen disclosed herein until the patient dies. In some embodiments, the patient is administered a treatment regimen disclosed herein until the patient withdraws consent to continued treatment. In some embodiments, the patient is administered a treatment regimen disclosed herein until the prostate cancer has been determined to be in remission. “Remission” is defined as a decrease in or disappearance of signs and symptoms of the cancer. In some embodiments, the patient is administered a treatment regimen disclosed herein for one or more treatment cycles of about 28 days.

In some embodiments, afuresertib, or a pharmaceutically acceptable salt thereof, is administered to the patient orally. In some embodiments, CFG920, or a pharmaceutically acceptable salt thereof, is administered to the patient orally. In some embodiments, the prednisone is administered to the patient orally.

In some embodiments, the dosage of: afuresertib, or a pharmaceutically acceptable salt thereof; CFG920, or a pharmaceutically acceptable salt thereof; or both can be decreased if the patient has been identified as exhibiting one or more symptoms associated with one or more treatment-emergent adverse events (TEAEs). The one or more treatment-emergent adverse events can include one or more of hyponatremia, hyperkalemia, hyperglycemia, hypomagnesemia, asthenia, fatigue, lethargy, insomnia, anemia, memory impairment, amnesia, skin infection, upper respiratory tract infection, pneumonia, blood alkaline phosphatase increased, back pain, bone pain, abdominal pain, constipation, dizziness, nausea, vomiting, diarrhea, dyspepsia, decreased appetite, dysphagia, dyspnea, eating disorder, pyrexia, weight loss, gastroesophageal reflux disease, gastrointestinal injury, thrombocytopenia, soft tissue necrosis, platelet count decrease, neutropenia, febrile neutropenia, odynophagia, pruritus, myalgia, stomatitis, peripheral neuropathy, rash, alopecia, sepsis, liver function test abnormalities, cardiac toxicity, ALT increase, arthralgia, AST increase, atrial fibrillation, herpes zoster, lipase increased, squamous cell carcinoma, dysuria, and urinary tract infection.

In some embodiments, the patient is additionally administered an androgen deprivation therapy. In some embodiments, the androgen deprivation therapy is a luteinizing hormone releasing hormone (LHRH) agonist or antagonist. In some embodiments, the patient remains on the androgen deprivation therapy throughout the course of the treatment methods of the present disclosure. In some embodiments, the patient has undergone surgical orchiectomy. In certain embodiments, the patient is administered an androgen deprivation therapy sufficient to maintain castration levels of serum testosterone, less than about 50 ng/dL serum testosterone levels or less than about 1.7 nmol/L serum testosterone levels.

When employed as pharmaceuticals, the compounds of the disclosure can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral, or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compounds of the disclosure or a pharmaceutically acceptable salt thereof, in combination with one or more pharmaceutically acceptable excipients. In making the compositions of the disclosure, the active ingredient is typically mixed with an excipient, diluted by an excipient or enclosed within such an excipient in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

The compositions can be formulated in a unit dosage form. The term “unit dosage forms” refers to physically discrete units suitable as unitary dosages for human patients and other mammals, each unit containing a predetermined quantity of active material calculated to produce the desired therapeutic effect, in association with a suitable pharmaceutical excipient.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

In some embodiments, afuresertib, or a pharmaceutically acceptable salt thereof, is formulated as part of a pharmaceutically acceptable composition further comprising one or more pharmaceutically acceptable excipients. In some embodiments, the pharmaceutical composition comprising afuresertib, or a pharmaceutically acceptable salt thereof, is suitable for oral administration. In some embodiments, the pharmaceutical composition comprising afuresertib or a pharmaceutically acceptable salt thereof, further comprises one or more of microcrystalline cellulose, mannitol, croscarmellose sodium and magnesium stearate. In some embodiments, the pharmaceutical composition comprising afuresertib or a pharmaceutically acceptable salt thereof is in the form of the following formulation:

| | Composition % |
|---|---|
| Component | by weight |
| Afuresertib HCl salt | 18.1 |
| Microcrystalline cellulose | 67.9 |
| Mannitol | 10.0 |
| Croscarmellose sodium | 27.8 |
| Magnesium stearate | 1.2 |
| Total unit dose | 100 |

In some embodiments, the afuresertib is formulated as a tablet having the following composition:

| | Quantity | |
|---|---|---|
| | mg/tablet | |
| | 50 mg Afuresertib | 75 mg Afuresertib |
| Component | (based on free base) | (based on free base) |
| Afuresertib HCl salt | 54.3 | 81.4 |
| Microcrystalline cellulose | 203.6 | 305.5 |
| Mannitol | 30.0 | 45.0 |
| Croscarmellose sodium | 8.3 | 12.5 |
| Magnesium stearate | 3.8 | 5.6 |
| Total unit dose | 300.0 | 450.0 |
| Opadry ® White aqueous | 6.0 to 12.0 | 9.0 to 18.0 |
| film coat | | |

In some embodiments, CFG920, or a pharmaceutically acceptable salt thereof, is formulated as part of a pharmaceutically acceptable composition further comprising one or more pharmaceutically acceptable excipients. In some embodiments, the pharmaceutical composition comprising CFG920, or a pharmaceutically acceptable salt thereof, is suitable for oral administration. In some embodiments, the pharmaceutical composition comprising CFG920, or a pharmaceutically acceptable salt thereof, further comprises prednisone. In some embodiments, the pharmaceutical composition comprising CFG920 or a pharmaceutically acceptable salt thereof, further comprises one or more of microcrystalline cellulose, mannitol, magnesium stearate, sodium starch glycolate and colloidal silicon dioxide. In some embodiments, the pharmaceutical composition comprising CFG920 or a pharmaceutically acceptable salt thereof is in the form of the following formulation:

| | Composition % |
|---|---|
| Component | by weight |
| CFG920-free base | 22.7 |
| Microcrystalline cellulose | 17.6 |
| Mannitol | 54.5 |
| Sodium starch glycolate (type A) | 3.6 |
| Magnesium stearate | 1 |
| Colloidal silicon dioxide | 0.45 |
| Total capsule fill weight | 100 |

In some embodiments, the CFG920 is formulated as a tablet having the following composition:

| | Quantity | |
|---|---|---|
| | mg/capsule | |
| Component | 25 mg CFG920 | 100 mg CFG920 |
| CFG920-free base | 25.0 | 100.0 |
| Microcrystalline cellulose | 19.4 | 51.6 |
| Mannitol | 60.0 | 184.0 |
| Sodium starch glycolate (type A) | 4.0 | 14.3 |
| Magnesium stearate | 1.1 | 3.4 |
| Colloidal silicon dioxide | 0.5 | 1.7 |
| Capsule fill weight | 110.0 | 355.0 |
| Empty capsule shell | 48.0 | 96.0 |
| Total capsule weight | 158.0 | 451.0 |

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles. Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional excipients. In some embodiments, ointments can contain water and one or more hydrophobic excipients selected from, for example, liquid paraffin, polyoxyethylene alkyl ether, propylene glycol, white Vaseline, and the like. Excipient compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the disclosure. The topical formulations can be suitably packaged in tubes of, for example, 100 g which are optionally associated with instructions for the treatment of the select indication.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous excipient prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present disclosure can vary according to, for example, the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration.

The compositions of the disclosure can further include one or more additional pharmaceutical agents such as a chemotherapeutic, steroid, anti-inflammatory compound, or immunosuppressant.

In certain embodiments, the active compounds may be prepared with an excipient that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

As used herein, the term “subject”, “individual” or “patient,” used interchangeably, refers to any animal, including mammals, preferably mice, rats, other rodents, rabbits, dogs, cats, swine, cattle, sheep, horses, or primates, and most preferably humans.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease. In some embodiments, the term “treating” or “treatment” refers to inhibiting or ameliorating the disease.

Provided herein are also methods of preventing a disease. For example, preventing a disease, condition or disorder in an individual who may be predisposed to the disease, condition or disorder but does not yet experience or display the pathology or symptomatology of the disease.

As used herein, “about” when referring to a measurable value such as an amount, a dosage, a temporal duration, and the like, is meant to encompass variations of ±10%. In certain embodiments, “about” can include variations of ±5%, ±1%, or ±0.1% from the specified value and any variations there between, as such variations are appropriate to perform the disclosed methods.

All compounds, and pharmaceutically acceptable salts thereof, can be found together with other substances such as water and solvents (e.g., in the form of hydrates and solvates) or can be isolated. In some embodiments, the compounds of the disclosure, or salts thereof, are substantially isolated. By “substantially isolated” is meant that the compound is at least partially or substantially separated from the environment in which it was formed or detected. Partial separation can include, for example, a composition enriched in the compounds of the disclosure. Substantial separation can include compositions containing at least about 50%, at least about 60%, at least about 70%, at least about 80%, at least about 90%, at least about 95%, at least about 97%, or at least about 99% by weight of the compounds of the disclosure, or salt thereof. Methods for isolating compounds and their salts are routine in the art.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, immunogenicity or other problem or complication, commensurate with a reasonable benefit/risk ratio.

The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the phrase “pharmaceutically acceptable excipient” refers to a pharmaceutically-acceptable material, composition, or vehicle, such as a liquid or solid filler, diluent, solvent, or encapsulating material. Excipients are generally safe, non-toxic and neither biologically nor otherwise undesirable and include excipients that are acceptable for veterinary use as well as human pharmaceutical use. In one embodiment, each component is “pharmaceutically acceptable” as defined herein. See, e.g., Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

It is appreciated that certain features of the present disclosure, which are, for clarity, described in the context of separate embodiments, can also be provided in combination in a single embodiment (while the embodiments are intended to be combined as if written in multiply dependent form). Conversely, various features of the present disclosure which are, for brevity, described in the context of a single embodiment, can also be provided separately or in any suitable subcombination.

As used herein, “QD” is taken to mean a dosage administered to the patient once-daily. “BID” is taken to mean a dosage administered to the patient twice per day.

An “adverse event” (AE) is defined as any untoward medical occurrence in a clinical study patient administered a medicinal product which does not necessarily have a causal relationship with this treatment.

The following abbreviations may be used herein:
AE=adverse event; CTCAE=Common Terminology Criteria for Adverse Events; DCR=disease control rate; DOR=duration of response; ECG=electrocardiogram; mCRPC=metastatic castration-resistant prostate cancer; ORR=overall response rate; OS=overall survival; PCWG3=Prostate Cancer Working Group 3; PSA=prostate-specific antigen; PTEN=phosphatase and tensin homolog; RECIST 1.1=Response Evaluation Criteria in Solid Tumors version 1.1; rPFS=radiological progression free survival.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. It is appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

Various modifications of the invention, in addition to those described herein, will be apparent to those skilled in the art from the foregoing description. Such modifications are also intended to fall within the scope of the appended claims. Each reference cited in the present disclosure, including all patent, patent applications, and publications, is incorporated herein by reference in its entirety.

### EXAMPLES

### Example A: Mouse Mini-PDX Model Study

### General Study Design

The objective of the study was to evaluate the in vivo therapeutic efficacy of CFG920 and afuresertib in MDX191210 Mini-PDX model. Tumor samples were taken from a 63-years old male patient, who was diagnosed with abiraterone resistant prostate carcinoma.

### Animals

Male Balb/c nude mice were purchased from Nanjing Biomedical Research Institute of Nanjing University (Nanjing, China, SCXK(Su)2018-0008), certification: 201806774. Species: Mus Musculus; Strain: Balb/c nude; Age: 6-8 weeks; Sex: male; Body weight: 20-25 g; Number of animals: 6 mice. Animals had free access to irradiation sterilized dry granule food during the entire study period. Animals had free access to sterile drinking water.

The mice were kept in a specific pathogen-free room at constant temperature and humidity with two animals in each cage. Housing conditions: Temperature: 20˜26° C.; Humidity: 40˜70%; Light cycle: 12 hours light and 12 hours dark. Cages were made of polycarbonate (325 mm×210 mm×180 mm). The bedding material was corn cob, changed twice per week. The identification labels for each cage contained the following information: number of animals, sex, strain, date received, treatment, study number, group number, and the starting date of the treatment. Animals were marked by ear coding.

### Mini-PDX Device

Mini-PDX capsule device is a modified microencapsulation and hollow fiber culture system (Onco Vee MiniPDX®, LIDE Biotech). The capsules are made of hollow fiber membrane with a pore size allowing passage of molecules less than 500 kDa. The fiber system delivers media to the cells in a manner similar to the delivery of blood through the capillary networks in vivo. Additional information regarding the operation of the Mini-PDX device can be found in Zhang, et al., Cancer Communications, 38, 60, 2018, which is incorporated by reference herein.

### Methods

Prostate tumor tissue was stored in a 10 cm petri dish in a biosafety cabinet. The tumor tissue was washed with Hank's balanced salt solution (HBSS) and non-tumor tissue and necrotic tumor tissue was removed. The tumor was cut into 1˜3 mm³ fragments using a scalpel, and then the minced tissue was transferred to a 50 ml conical vial. Collagenase solution (10×) was added to the vial to a final concentration of 1×. The tube was closed with a cap and the cap was wrapped with parafilm to prevent potential bacteria/yeast contamination. The tube was placed on its side in a 37° C. shaker at 200 rpm speed for 1-2 hours. The tube was centrifuged at 500×g at room temperature for 5 min to pellet the cells. The pellet was resuspended in 200 μl HBSS and immune cells and stromal cells were depleted using anti-fibroblast microbeads (Miltenyi, cat: 130-050-601), anti-CD45 microbeads (Miltenyi, cat: 130-045-801), LS column (Miltenyi, cat: 130-042-401) and QuadroMACS magnet (Miltenyi, cat: 130-091-051). The remaining tumor cells were collected and washed with HBSS and filled into Mini-PDX capsule devices (OncoVee MiniPDX®, LIDE Biotech). The capsules were implanted subcutaneously into both flanks of Balb/c nude mice via a small skin incision, with 3 capsules per mouse for the Mini-PDX efficacy study. Treatment periods for these studies were 7 days. At the termination of the study, all mice were euthanized, the implanted capsules were removed and tumor cell proliferation was evaluated using the CellTiter Glo Luminescent Cell Viability Assay kit (G7571, Promega, Madison, WI, US) as instructed by the manufacturer. Luminescence was measured in terms of relative luminance unit (RLU) using a spectrophotometer (SpectraMax M3, Molecular Devices, Sunnyvale, CA, US). Relative Viability (%) was calculated using the formula:

Tumor Relative Proliferation values (%)=(Mean RLU of the treatment group on day 7−Mean RLU on day 0)/(Mean RLU of the vehicle group on day 7−Mean RLU on day 0)× 100%

The test drug administration and the number of animals in each study group are summarized in the following experimental design (Table 1).

TABLE 1
Groups and Treatment

| Group | N | Treatment | Dose (mg/kg) | Dosing Route | Schedule |
|---|---|---|---|---|---|
| 1 | 6 | Vehicle | 0 | p.o. | QD × 7 |
| 2 | 6 | afuresertib | 75 | p.o. | QD × 7 |
| 3 | 6 | afuresertib + | 75 (afuresertib) + | p.o. + p.o. | QD × 7 for afuresertib; |
| | | CFG920 | 300 (CFG920) | | BID for CFG920 |

Note:
N: number of Mini-PDX device equipped test subjects; QD: once every day; BID: twice every day.
p.o.: oral administration

TABLE 2
Anti-tumor efficacy study of CFG920 and afuresertib
in the treatment of MDX191210 MiniPDX model

| | | Cell Viability (CTG Units) | | | Relative | Average |
|---|---|---|---|---|---|---|
| | | (Mean ± SEM)/ | (Mean ± SEM)/ | | Proliferation | RCBW |
| Group | N | Day 0 | Day 0 | P Value | (%) | (%)/Day 6 |
| Vehicle | 6 | 3981 ± 683 | 42459 ± 4790 | — | 100.00 | 4.32 |
| afuresertib | 6 | | 24516 ± 2133 | 0.00065* | 53.37 | −8.73 |
| afuresertib + | 6 | | 14701 ± 1790 | 0.0003* | 27.86 | −18.18 |
| CFG920 | | | | | | |

Note:
*P < 0.01, compared with vehicle control group by student's t test

TABLE 3
Test compounds and formulation and preparation

| | | Concentration | |
|---|---|---|---|
| Compounds | Preparation | (mg/mL) | Storage |
| Vehicle | 0.5% HPMC + 0.2% | — | 4° C. |
| CFG920 | TWEEN 80 Dissolve | 30 | 4° C. |
| | 137.76 mg in 4.5 mL 1% | | |
| | TWEEN 20/0.5% | | |
| | hydroxyethyl cellulose | | |
| | (1:1), and mix well | | |
| afuresertib | Dissolve 34.44 mg in | 7.5 | 4° C. |
| | 4.5 mL 1% w/v methylcellulose | | |
| | in water and mix well | | |

### Observation

During the study, the care and use of animals were conducted in accordance with the regulations of the Association for Assessment and Accreditation of Laboratory Animal Care (AAALAC). After Mini-PDX device inoculation, the animals were checked daily for morbidity and mortality. At the time of routine monitoring, the animals were checked for any effects of treatments on normal behavior such as mobility, visual estimation of food and water consumption, body weight gain/loss, eye/hair matting and any other abnormal effect.

### Endpoints

Tumor relative proliferation rate (%) was used as the indication of anti-tumor activity. Tumor relative proliferation rate (%)=Vt₇/Vc₇×100% (Vt₇: the cell viability of treatment group on day 7; Vc₇: the cell viability of Vehicle Control group on day 7).

The body weight of mice was measured every day, and relative change of bodyweight was calculated for each mouse according to the following formula: RCBW(%)=(BWᵢ−BW₀)/BW₀×100, BWᵢ is the body weight after treatment on a given day, BW₀ is the bodyweight on the first day of treatment.

### Results

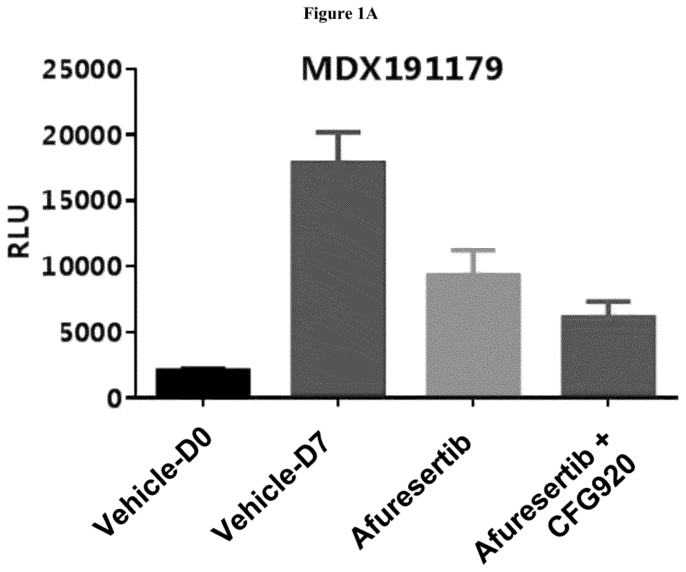

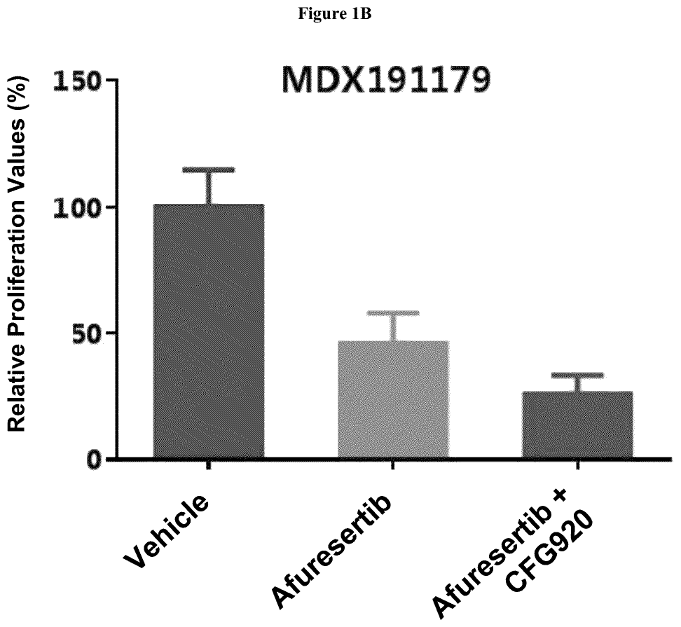

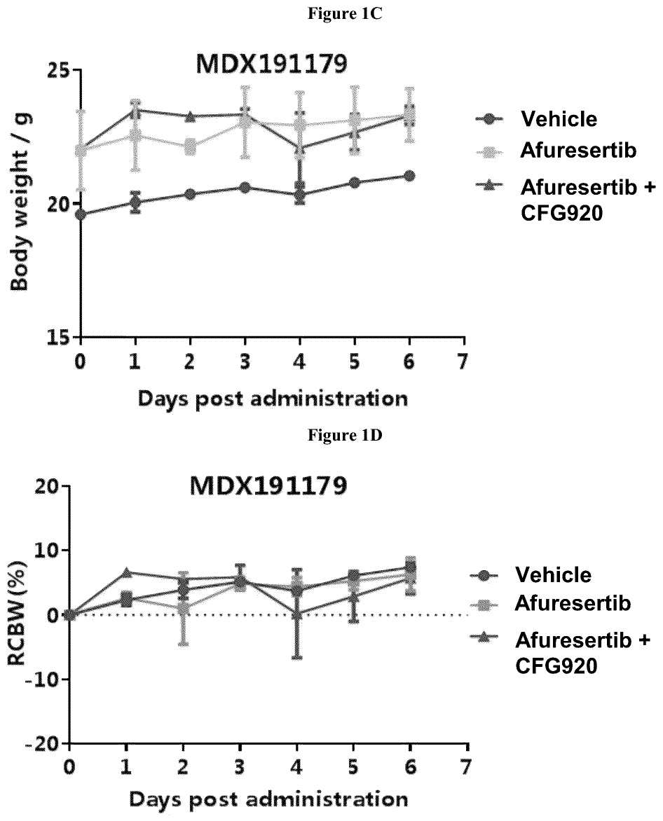

The objective of the efficacy study was to evaluate the therapeutic efficacy of CFG920 and afuresertib in MDX191210 Mini-PDX model. At the termination, the CTG values of the treatment groups of vehicle control, afuresertib 75 mg/kg and afuresertib 75 mg/kg+CFG920 300 mg/kg were 42459±4790, 24516±2133 and 14701±1790 respectively (FIG. 1A). Tumor relative proliferation values (%) of afuresertib 75 mg/kg and afuresertib 75 mg/kg+CFG920 300 mg/kg groups were 53.37% and 27.86% (FIG. 1B). When compared to vehicle control, afuresertib 75 mg/kg and afuresertib 75 mg/kg+CFG920 300 mg/kg treatment resulted in statistically significant decrease in MDX191210 tumor cell viability as monitored by the CTG assay (P<0.01). The data suggested that afuresertib 75 mg/kg and afuresertib 75 mg/kg+CFG920 300 mg/kg treatment inhibited the proliferation of MDX191210 tumor cells. During this study, afuresertib 75 mg/kg and afuresertib 75 mg/kg+CFG920 300 mg/kg treatment groups did not result in significant body weight loss (body weight loss<10%) (FIG. 1C). Body weight loss of one mouse>15% in afuresertib 75 mg/kg+CFG920 300 mg/kg group at day 5 and day 6, could be attributed to individual differences. The data suggested that afuresertib 75 mg/kg or afuresertib 75 mg/kg+CFG920 300 mg/kg was tolerated well by the animals (Table 2).

In summary, afuresertib 75 mg/kg and afuresertib 75 mg/kg+CFG920 300 mg/kg treatment groups showed significant antitumor activity in MDX191210 MiniPDX model and the treatment was well-tolerated.

### Example B: Dose-Escalation and Efficacy Study of CFG920 and Prednisone Plus Afuresertib in Patients with Metastatic Castration-Resistant Prostate Cancer Following Standard of Care Treatment

### Overall Design

The Phase I part of the study is a dose-escalation study to identify the recommended Phase II dose (RP2D) of the combined therapy of CFG920 and prednisone+afuresertib in metastatic castrate resistant prostate cancer (mCRPC) patients who progressed on, or are intolerant of, two prior treatments of any anti-androgen therapy (such as abiraterone, enzalutamide, apalutamide, or any other androgen receptor (AR) antagonists that are approved later), or one of the above anti-androgen treatment plus one chemotherapy selected from docetaxel and cabazitaxel regardless of the PTEN status. The Phase II part of the study assesses the preliminary efficacy and safety of the combined therapy of CFG920 and prednisone+afuresertib compared to afuresertib monotherapy, in mCRPC patients with PTEN loss who progressed on, or are intolerant of, two prior treatments of any anti-androgen (as described above), or one of the above anti-androgen treatment plus one of the chemotherapy selected from docetaxel and cabazitaxel.

Phase I: The study employs a 3+3 design in each dose-escalation stage. The maximum tolerated dose (MTD) evaluation or RP2D is based on the observed profile of safety, PK, and PD in patients. The dose-escalation decision is made based on the observed safety profiles, PK, and PD in that particular dose level. Three patients are enrolled at the starting dose of the combinational therapy.

Dose-escalation is stopped, and the cohort is expanded to six patients if a patient experiences a dose-limiting toxicity (DLT) as described in the table of dose escalation guidelines below. The starting dose was selected based on previous phase I/II monotherapy study of CFG920, prednisone and afuresertib in different cancer indications, such as prostate cancer, ovarian cancer or gastric cancer. The recommended combined dose-escalation are as follows, except on Cycle 1 Day 1 (C1D1) where all patients receive only a single dose of CFG920 and prednisone at the specified doses along with afuresertib once daily (QD).

TABLE 4A
Dose escalation guidelines

| Number of Subjects | |
|---|---|
| with DLT | Action |
| 0 out of 3 subjects | Escalate to next dose level |
| 1 out of 3 subjects | Accrue three additional evaluable subjects at |
| | current dose level for a total of six evaluable |
| | subjects. |
| 1 out of 6 subjects | Escalate to next dose level |
| 2 or more subjects | MTD has been exceeded. |
| in a dosing cohort | |
| (up to 6 subjects) | |

TABLE 4B
Dosage

| COHORT | DOSAGE |
|---|---|
| Cohort 3 | CFG920 100 mg BID + prednisone 5 mg BID + afuresertib 125 mg QD |
| | (CFG920 1 × 100 mg capsules and prednisone 1 × 5 mg tablet BID, and |
| | afuresertib 1 × 50 mg plus 1 × 75 mg tablets QD) |
| Cohort 2**** | CFG920 75 mg BID + prednisone 5 mg BID + afuresertib 125 mg QD |
| | (CFG920 3 × 25 mg capsules and prednisone 1 × 5 mg tablet BID, and |
| | afuresertib 1 × 50 mg plus 1 × 75 mg tablets QD) |
| Cohort 1 | CFG920 75 mg BID + prednisone 5 mg BID + afuresertib 100 mg QD |
| (Starting | (CFG920 3 × 25 mg capsules and prednisone 1 × 5 mg tablet BID, and |
| Dose) | afuresertib 2 × 50 mg tablets QD) |
| Cohort −1* | CFG920 50 mg BID + prednisone 5 mg BID + afuresertib 100 mg QD |
| | (CFG920 2 × 25 mg capsules and prednisone 1 × 5 mg tablet BID, and |
| | afuresertib 2 × 50 mg tablets QD) |
| Cohort −2A** | CFG920 50 mg BID + prednisone 5 mg BID + afuresertib 125 mg QD |
| | (CFG920 2 × 25 mg capsules and prednisone 1 × 5 mg tablet BID, and |
| | afuresertib 1 × 50 mg plus 1 × 75 mg tablet QD) |
| Cohort −2B*** | CFG920 50 mg BID + prednisone 5 mg BID > afuresertib 75 mg QD |
| | (CFG920 2 × 25 mg capsules and prednisone 1 × 5 mg tablet BID, and |
| | afuresertib 1 × 75 mg tablets QD) |

*If >33.3% patients in the cohort 1 (CFG920 75 mg BID + prednisone 5 mg BID + afuresertib 100 mg QD) and >33.3% patients in the expanded cohort 1 experience DLTs, the combined dose will be reduced to (CFG920 50 mg BID + prednisone 5 mg BID + afuresertib 100 mg QD) i.e. Cohort −1
**If <33.3% patients in the cohort −1 (CFG920 50 mg BID + prednisone 5 mg BID + afuresertib 100 mg QD) and <33.3% patients in the expanded cohort −1 experience DLTs, the patient's next dose level would be (CFG920 50 mg BID + prednisone 5 mg BID + afuresertib 125 mg QD) i.e. Cohort −2A
***If >33.3% patients in the cohort −1 (CFG920 50 mg BID + prednisone 5 mg BID + afuresertib 100 mg QD) and >33.3% patients in the expanded cohort −1 experience DLTs, the patient's next dose level would be (CFG920 50 mg BID + prednisone 5 mg BID + afuresertib 75 mg QD) i.e. Cohort −2B
****If >33.3% patients in the cohort 2 (CFG920 75 mg BID + prednisone 5 mg BID + afuresertib 125 mg QD) and >33.3% patients in the expanded cohort 2 experience DLTs, the combined dose will be reduced to (CFG920 50 mg BID + prednisone 5 mg BID + afuresertib 125 mg QD) i.e. Cohort −2A
If >33.3% patients in the cohort −2B and >33.3% in the combined expanded cohort −2B (CFG920 50 mg BID + prednisone 5 mg BID + afuresertib 75 mg QD) experience DLTs, the study will be temporarily halted and the SRC, consisting of the study medical advisor/monitor and the investigators, will have an ad hoc meeting to decide how best to proceed.

There are 3 combined dose de-escalation levels Cohort-1 (CFG920 50 mg BID+prednisone 5 mg BID+afuresertib 100 mg QD); Cohort-2A (CFG920 50 mg BID+prednisone 5 mg BID+afuresertib 125 mg QD) and Cohort-2B (CFG920 50 mg BID+prednisone 5 mg BID+afuresertib 75 mg QD).

A treatment Cycle will consist of 28 days. The DLTs will be divided into hematologic DLTs and non-hematologic DLTs. A DLT is defined as an adverse event (AE) or abnormal laboratory value assessed as unrelated to disease, disease progression, intercurrent illness, or concomitant medications and at least possibly related to CFG920+prednisone+afuresertib treatment that occurs within the first Cycle (28 days) of the Phase I period and meets any of the criteria included below:

TABLE 4C

| Dose-Limiting | | |
|---|---|---|
| Toxicity | Common Terminology Criteria for | |
| (DLT) Type | AEs (CTCAE v5.0) | Details |
| Hematologic | Neutropenia Grade 3 | Absolute neutrophil count <1.0 × |
| | | 10³/L; >5 days |
| | Neutropenia Grade 4 | Any duration |
| | Febrile neutropenia Grade 3 and 4 | Absolute neutrophil count <1.0 × |
| | | 10⁹/L, fever ≥38.3° C. |
| | Thrombocytopenia Grade 3 | With clinical evident bleeding |
| | Thrombocytopenia Grade 4 | With or without bleeding |
| Non- | Laboratory abnormalities that satisfy | Any duration |
| Hematologic | Hy's Law (ALT or AST elevation >3× ULN, | |
| | total bilirubin elevation >2× ULN, | |
| | absence of initial findings | |
| | of cholestasis [i.e., absence of | |
| | elevation of alkaline phosphatase to | |
| | >2× ULN] and no other reason can | |
| | be found to explain the combination | |
| | of increased ALT/AST and total | |
| | bilirubin). | |
| | Any clinically significant, treatment | Lasting >7 days |
| | related grade ≥3 non-hematologic | Or medical intervention is required |
| | laboratory abnormality | to treat the patient or abnormality |
| | | leads to hospitalization |
| | Any grade 3 study drug related AEs | Lasting >3 days (skin rash & |
| | | diarrhea >5 days) despite optimal |
| | | supportive care. (Grade 3 fatigue |
| | | will NOT be classified as DLT, |
| | | irrespective of duration). |
| | Any grade 4 study drug related AE | Any duration |
| | Any study drug related toxicity which | Lasting >2 weeks |
| | causes study drug hold | |

In addition, the incidence of≥Grade 3 anemia, thrombocytopenia, fatigue, nausea and vomiting, as well as hyperkalemia/hypokalemia, hypernatremia/hyponatremia, and hypertension/hypotension are considered AEs of special interest (AESI) and their incidences will be analyzed separately.

Patients who discontinue the study due to any reasons other than DLT and who have received <21 days or missed >25% of the planned doses of either CFG920 and prednisone or afuresertib during the first treatment Cycle (28 days) of Phase I period are replaced.

The PK of CFG920 and prednisone+afuresertib treatment is assessed based on plasma levels of CFG920 and afuresertib obtained at different time points on C1D1 and C1D15, and pre-dose measurements on Day 1 of subsequent Cycles. The PD of CFG920 and prednisone+afuresertib is assessed by the periodic measurement of certain adrenal hormones, testosterone, and blood phosphorylated glycogen synthase kinase 3 beta (pGSK3β) levels at specified time points.

The MTD is defined as the highest combined drug dosage at which ≤33% of patients experience DLT in the first Cycle of combined therapy of CFG920 and prednisone+afuresertib. The highest combinational dose, at which 6 patients have been treated and ≤33% patients experienced a DLT, move forward to the Phase II cohort as the RP2D after review by the SRC.

The preliminary efficacy of the combined therapy of CFG920 and prednisone+afuresertib in mCRPC patients is assessed in Phase I as described in the objective and endpoint section.

Phase II: Once the RP2D of CFG920 and prednisone+afuresertib has been established, a cohort consisting of 50 mCRPC patients with PTEN loss who have progressed on, or are intolerant of, two prior standard treatments of any anti-androgen (such as abiraterone, enzalutamide, apalutamide, or any other AR antagonists that are approved later), or one of the above anti-androgen treatment plus one of the chemotherapy from docetaxel or cabazitaxel are enrolled in the Phase II to evaluate the preliminary efficacy and safety of CFG920 and prednisone+afuresertib at the RP2D and afuresertib 150 mg QD monotherapy. The eligible patients are randomized with a ratio of 1:1 into two treatment groups, CFG920 and prednisone BID+afuresertib QD and afuresertib monotherapy. The preliminary efficacy of CFG920 and prednisone+afuresertib and afuresertib monotherapy is assessed by the measurement of radiological progression free survival (rPFS), overall response rate (ORR), duration of response (DOR), disease control rate (DCR), overall survival (OS), prostate-specific antigen (PSA) monitoring according to the Prostate Cancer Working Group 3 (PCWG3) and radiographic tumor assessments (bone lesions based on PCWG3 and other lesions based on Response Evaluation Criteria in Solid Tumors version 1.1 [RECIST 1.1]).

In the whole study period, the safety and tolerability of the combination therapy and monotherapy is closely monitored in mCRPC patients.

### Number of Patients

Phase I: If the maximum of 4 dose-escalation cohorts moving up and 2 cohorts moving down require the enrollment of up to 6 patients/each cohort (6 patients are needed for RP2D or MTD decision), the study can enroll a maximum of 24 patients in the Phase I. The number of enrolled patients may exceed this number if more doses levels are explored. Additional patients may be enrolled to replace dropouts other than the patients who discontinue due to a DLT.

Phase II: In the Phase II part, 50 patients are enrolled and randomized in a 1:1 ratio to the 2treatment groups, 25 patients per group, to evaluate preliminary efficacy of the combined therapy with CFG920 and prednisone+afuresertib and afuresertib monotherapy for mCRPC patients; randomization is stratified by prior chemotherapy (yes/no). Replacement is not allowed in the Phase II part.

### Treatment Groups and Duration

Study treatments are administered in DLT-observation Cycles of 28 days each. CFG920 is administered orally as 25 mg and/or 100 mg capsules. Afuresertib is administered orally as 50 mg and/or 75 mg tablets. Prednisone is administered orally as 5 mg tablets. CFG920 and prednisone are administered BID, except on Cycle 1 Day 1 of Phase I where only a single morning dose of each drug is administered. Afuresertib is administered QD (at the same time as the morning CFG920 dose, for CFG920 and prednisone+afuresertib treatment).

Patients continue to receive luteinizing hormone releasing hormone (LHRH) agonists or antagonists per labeled directions for administration. The adrenal insufficiency and aldosterone excess related laboratory tests and AESIs are closely monitored, and necessary clinical management is applied by investigators based on clinical laboratory tests results.

### Study Duration

The main data analysis and reporting are performed when all patients have completed at least 6 months of treatment, progressed, dropped out from the study (due to reasons such as ICF withdrawal, investigator's decision or non-compliance), or died for any reason. The end of study for Phase II is defined as the time when at least 90% of patients in the Phase II study have progressed, dropped out from the study (due to reasons such as ICF withdrawal, investigator's decision or non-compliance), or died for any reason.

### Study Population

Study patients are selected from males ≥18 years of age with documented histological or cytological evidence of adenocarcinoma of the prostate (excluding neuroendocrine differentiation or small cell histology). Patients must have radiographic evidence of metastatic disease based on the ‘Guidelines of American Urological Association for Prostate Cancer’ before study enrollment. (Lowrance WT, et al. Advanced Prostate Cancer: AUA/ASTRO/SUO Guideline PART I. J Urol. 2021 January:205(1): 14-21), and be able to provide tumor biopsy samples for PTEN immunohistochemistry (IHC) staining. A valid PTEN IHC result must be collected within 2 months of screening visit and be confirmed by central laboratory testing (participants with an “invalid” or “failed” PTEN IHC result are not permitted to be enrolled). Patients must have progressive disease based on the PCWG3 criteria. PCWG3 criteria includes 1) Patients who progressed based solely on total PSA rising, should have had a sequence of rising values on 3 consecutive occasions of at least 1-week intervals (if the third measurement is not greater than the second measurement, a fourth measurement at least a week apart must be taken and must be greater than the second measurement) and should have 2.0 ng/mL minimum level for entry; 2) Patients who have documented disease progression per RECIST 1.1 are eligible independent of PSA; 3) Patients with bone only progression according to PCWG3 (ie, bone scan showing appearance of ≥2 new lesions). Patients must have had a prior PSA response, followed by documented PSA progression on prior hormone treatment. Patients must have castration levels of testosterone (<50 ng/dL or 1.7 nmol/L). Patients must have undergone androgen deprivation therapy (ADT), such as orchiectomy, or have been on LHRH agonists or antagonists, for at least 3 months prior to study enrollment. Patients on LHRH agonists/antagonists must remain on these agents for the duration of the study. Patients must have an Eastern Cooperative Oncology Group (ECOG) performance status of ≤1. Patients must have adequate hematopoietic function by local laboratory within the 28 days before enrollment, as evidenced by: Absolute neutrophil count ≥1,500/μL, Platelet count ≥75,000/μL, Hemoglobin ≥9 g/dL. Total serum bilirubin ≤1.5×ULN within the 28 days before enrollment (in patients with known Gilbert' s syndrome, total bilirubin ≤3×ULN with direct bilirubin ≤1.5×ULN). Aspartate aminotransferase and alanine aminotransferase ≤2.5×ULN except for patients with tumor involvement of the liver who must have AST and ALT ≤5×ULN within the 28 days before enrollment. Patients must have adequate renal function as evidenced by a serum creatinine of ≤1.5×the ULN for the reference laboratory or creatinine clearance ≥50 mL/min within the 28 days before enrollment (calculated from Cockcroft-Gault formula or 24-hour urine collection). Serum potassium ≥3.5 mmol/L and <ULN within the 28 days before enrollment. Fasting plasma glucose [fasting is defined as no caloric intake for at least 8 hours]: ≤126 mg/dL or ≤7.0 mmol/L for those patients without a pre-existing diagnosis of Type 2 diabetes mellitus; ≤167 mg/dl or ≤9.3 mmol/L for those patients with a pre-existing diagnosis of Type 2 diabetes mellitus, Glycosylated haemoglobin (HbA1C) ≤8.0%.

For Phase I, patients must have mCRPC which has progressed or are intolerant after receiving two prior treatments (in some instances at least 1 prior treatment) of any anti-androgen (such as abiraterone, enzalutamide, apalutamide, or any other AR antagonists that are approved later), or one of the above anti-androgen treatments plus one of the chemotherapies from docetaxel or cabazitaxel. Patients must have at least 3 weeks of treatment of any antiandrogen and/or completed at least 4 Cycles of docetaxel or cabazitaxel treatment before their screening visit.

For Phase II, patients must have mCRPC progressed or are intolerant after receiving only 2 prior treatments of any anti-androgen (such as abiraterone, enzalutamide, apalutamide, or any other AR antagonists that are approved later), or one of the above anti-androgen treatments plus one of the chemotherapies from docetaxel or cabazitaxel. Patients must have at least 3 weeks of treatment of any antiandrogen and/or completed at least 4 Cycles of docetaxel or cabazitaxel treatment before their screening visit. Only 2 prior treatments are allowed because this combination therapy is targeting the third line therapy for mCRPC.

Patients are excluded if they have undergone major surgery within 28 days before study treatment or if they have been treated with any of the following: second-line ADT (including but not limited to ketoconazole and amino glutethimide) within 6 weeks before enrollment; sipuleucel-T (Provenge®) treatment within 3 months of enrollment; antiandrogens such as flutamide (EULEXIN®), bicalutamide (CASODEX®), or nilutamide (NILANDRON®) within 6 weeks prior to enrollment; 5-alpha reductase inhibitors such as finasteride (PROSCAR®, PROPECIA®), or dutasteride (AVODART®) within 3 months of enrollment; Radium Ra 223 dichloride (XOFIGO®) or Samarium Sm 153 lexidronam (QUADRAMET®) within 3 months prior to enrollment; corticosteroids or another immunosuppressive agent, other than daily use of up to 10 mg prednisone (or equivalent) or low-dose steroid for the control of nausea and vomiting, topical steroid, or inhaled steroid use; potassium-wasting diuretics; patients who have received any investigational agent beyond those indicated for the treatment of prostate cancer within 5 half-lives of the agent; if the half-life of the agent is not known, the patients must be off investigational therapy for 4 weeks prior to enrollment (whichever is shorter of the two should be preferred); patients who have received palliative and other radiotherapy for the target lesion within 4 weeks of study enrollment; patients with symptomatic or known central nervous system metastases from prostate cancer or who are at high risk for spinal cord compression; patients with a history of hypothalamus, pituitary or adrenal insufficiency; patients with diabetes mellitus that require insulin at study enrollment; history of another primary malignancy that is currently clinically significant or currently requires active intervention; inadequately controlled hypertension (eg, systolic blood pressure ≥160 mmHg or diastolic blood pressure ≥95 mmHg) or hypotension (eg, systolic blood pressure ≤80 mmHg or diastolic blood pressure ≤50 mmHg) after up to 3 measurements with at least 5 minutes apart during 28 days before study enrollment; patients with active cardiac disease or a history of cardiac dysfunction including any of the following: a. severe or unstable angina pectoris or acute coronary syndrome or stroke within 6 months prior to study enrollment, b. symptomatic pericarditis, c. documented myocardial infarction or arterial thrombotic events within 6 months prior to study enrollment, d. history of documented congestive heart failure (New York Health Association functional classification III to IV), e. documented history of cardiomyopathy, f. known left ventricular ejection fraction <50% as determined by multiple gated acquisition scan or echocardiogram within 28 days prior to enrollment, g. history of clinically significant cardiac arrhythmias, as determined by the investigator; patients with a Fridericia-corrected QT (QTcF) interval of >450 msec on the screening electrocardiogram (ECG) (using the QTcF formula), has a short/long QT syndrome, or history of QT prolongation/Torsades de Pointes; patients with a history of an active infection (viral, bacterial, or fungal) requiring systemic therapy within 10 days before enrollment, including but not limited to tuberculosis; patients who have active human immunodeficiency virus (HIV), hepatitis B, or hepatitis C infections; patients that are currently receiving treatment with drugs known to be moderate or strong inhibitors or inducers of isoenzyme CYP1A (including but not limited: a-Naphthoflavone, Furafylline, Omeprazole, Lansoprazole) and isoenzyme CYP3A (including but not limited: Itraconazole, Ketoconazole, Azamulin, Troleandomycin, Verapamil, Rifampicin). The patients must have discontinued moderate or strong inducers for at least 2 weeks prior to study enrollment and must have discontinued moderate or strong inhibitors for at least 1 week before study enrollment. Spironolactone, Strong bile salt export pump (BSEP) inhibitors, grapefruit juice, herbal medicines such as St. John's wort, Kava, ephedra, gingko biloba, dehydroepiandrosterone, yohimbe, saw palmetto and ginseng should be discontinued; sexually active males not willing to use a condom during the whole course of the study and for 16 weeks after stopping treatment. Male patients must not father a child in this period. A condom is required to be used also by vasectomized men as well as during intercourse with a male partner in order to prevent delivery of the drug via seminal fluid; patients with any other medical, psychiatric, or social condition, including substance abuse, which in the opinion of the investigator, would preclude participation in the study; patients with a history of upper gastrointestinal bleeding or uncontrolled peptic disease in the previous 3 months which in Investigator's opinion may impact patient's participation in the trial; patients who have previously received AKT or PI3 kinase pathway or mTOR inhibitors.

### Statistical Methods

There is no hypothesis testing performed for the Phase I part of the study. Accordingly, the approach to statistical analysis is descriptive. For the Phase II part of the study, the following primary analysis is conducted:

The Kaplan-Meier method is used to estimate the median rPFS and provide the one-sided 90% Brookmeyer-Crowley Confidence Interval (CI) for the combination treatment group and the afuresertib single agent treatment group, respectively. A median rPFS ≥5.5 months with associated 1-sided 90% CI limit >3.5 months is considered a clinically meaningful improvement. No statistical hypothesis testing is performed for the comparison between treatment groups. For exploratory purposes, the Cox proportional hazard regression model is applied to analyze the rPFS of the 2 treatment groups with treatment and prior chemotherapy (yes/no) as the independent variables.

### Safety Assessments

Safety assessments include AEs (including DLTs), laboratory parameters, vital signs (pulse, blood pressure, respiratory rate, and temperature), height and weight, physical examination, and ECG.

### Efficacy Assessments

Treatment efficacy is determined for the combined therapy (Phase I and II) and afuresertib monotherapy (Phase II only) based on the following criteria: radiological progression free survival; overall objective response rate (Phase II only); overall survival (phase II only); duration of response; disease control rate (Phase II only); PSA levels collected at pre-specified time points; PSA response and time to PSA progression; and radiographic tumor assessment at pre-specified time points.

Anti-tumor activity is classified as Complete Response (CR), Partial Response (PR), Progressive Disease (P.D.), and Stable Disease (S.D.) following the instructions in RECIST 1.1 and PCWG3 based on the response evaluation of targeted and non-targeted lesions. The definitions of responses for targeted lesions are:
Complete Response (CR): Disappearance of all target lesions. Any pathological lymph nodes (whether target or non-target) must have reduction in short axis to <10 mm.
Partial Response (PR): At least a 30% decrease in the sum of diameters of target lesions, taking as reference the baseline sum diameters.
Progressive Disease (P.D.): At least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progression).
Stable Disease (S.D.): Neither sufficient shrinkage to qualify for PR nor sufficient increase to qualify for PD, taking as reference the smallest sum diameters while on study. Radiological progression free survival (rPFS) is measured as the time from initiation of therapy until disease progression, according to RECIST 1.1 (Appendix 6) and/or PCWG3 criteria, or death from any cause, whichever occurs first. Overall survival (OS) is measured as the time from initiation of therapy until death from any cause. Objective response rate (ORR) is the proportion of patients that achieve a best overall response (BOR) of confirmed CR or PR. Disease Control Rate (DCR) is the proportion of patients that achieve a BOR of confirmed CR, PR or SD. Duration of overall response is the time from date of first determination of response (CR or PR) to first date that recurrent or progressive disease is objectively documented.

PSA related efficacy is determined by obtaining a sequence of values at a minimum of 1-week intervals. PSA response is defined as a ≥50% reduction in PSA from baseline and can be confirmed by subsequent two PSA evaluations with a minimum of 3-week intervals. Secondary PSA response is defined as a ≥30% reduction in PSA from baseline to ≥12 weeks after study treatment and can be confirmed by the reexamination 4 weeks later.

### Selection and Timing of Doses

A single dose of CFG920 and prednisone is administered on Cycle 1 Day 1. Starting on Day 2 of Cycle 1 (after collection of the 24-hr PK sample) and throughout each treatment Cycle of the study, CFG920 is given orally, BID, with approximately 12 hours between each dose. Prednisone is administered at the same time as CFG920, given orally, BID. Afuresertib is administered once daily, at the same time as the morning dose of CFG920.

The capsules are ingested whole with 200 mL water and should not be chewed or opened. Doses of CFG920 are administered in the fasted state, at least 1 hour before or 2 hours after a meal. If the patient vomits, no re-dosing is allowed before the next scheduled dose.

On Cycle 1 Day 13 of Phase I, the site staff contact patients to remind them: that the BID dosing schedule should be maintained as close to the 12-hour dosing schedule as possible; on Cycle 1 Days 14 and 15, they should maintain an adequate period of fasting around the times of CFG920 dosing; and that they need to report to the site at the appropriate time on the morning of Cycle 1 Day 15 so that the pre-dose sample can be drawn and the morning doses administered at the scheduled time on that day. Dose dates and times for the following days are recorded: Cycle 1 Day 1, Cycle 1 Day 2 (morning doses), Cycle 1 Day 14 (morning and evening doses), Cycle 1 Day 15 (morning and evening doses), and Cycle 1 Day 16 (morning doses). For other BID dosing days, if the second dose is not taken 12 hours (±2 hours) after the first dose, the second dose is skipped. Any missed or skipped doses between Cycle 1 Day 1 and Cycle 1 Day 15 are recorded. The date and time of the patient's most recent ingestion of food prior to dose administration, as well as the date and time of the patient's next ingestion of food after dose administration are also recorded for the Cycle 1 Day 1 and Cycle 1 Day 15 morning doses.

### Warnings and Precautions

Based on animal study results, preliminary clinical safety data, and clinical data reported for abiraterone, potential toxicities to patients administered CFG920 and afuresertib are summarized below, respectively. During the clinical evaluation of CFG920 and afuresertib, patients will be monitored for AEs and laboratory test result changes to ensure their safety.

Potential toxicity in patients with CFG920 includes: hematologic toxicity; hyperglycemia; hepatic effects; decreased cortisol and related effects; reproductive organ changes; and clinical laboratory changes. Potential toxicity in patients with afuresertib includes: gastrointestinal toxicity; endocrine/metabolic toxicity; hepatic toxicity; dermatologic toxicity; and thyroid gland toxicity.

### Concomitant Therapy

Patients must have undergone orchiectomy or have been on LHRH agonists/antagonists for at least 3 months prior to enrollment. Patients on LHRH agonists/antagonists must remain on these agents for the duration of the study.

In general, the use of any concomitant medication/therapy, including over-the-counter medications deemed necessary for the care of the patient, is permitted during the study and properly captured in the electronic case report form (eCRF).

Patients who have received antiandrogens such as flutamide (EULEXIN®), bicalutamide (CASODEX®), or nilutamide (NILANDRON®) for >3 months have to be off treatment for 6 weeks before enrolment and demonstrate a continued rise in PSA after withdrawal. Patients on antiandrogens for ≤3 months must be off medication for 2 weeks.

Patients who have received radium ra 223 dichloride (XOFIGO®) must be off therapy for 7 weeks prior to enrollment or samarium sm 153 lexidronam (QUADRAMET®) must be off therapy for at least 2 weeks prior to study enrollment.

Patients who are currently receiving treatment with drugs known to be moderate or strong inhibitors/inducers of isoenzyme CYP1A (including but not limited: a-naphthoflavone, furafylline, omeprazole, lansoprazole) and isoenzyme CYP3A (including but not limited: itraconazole, ketoconazole, azamulin, troleandomycin, verapamil, rifampicin). These medicines must have discontinued strong inducers for at least 2 weeks and must have discontinued strong inhibitors for at least 1 week before the treatment is initiated.

Concomitant use of bisphosphonates and other bone supportive agents is allowed if the dose and renal function have been stable for at least 12 weeks before the enrollment and no related ≥Grade 2 side effects are present for at least 4 weeks prior to study drug treatment.

Concomitant therapy with fibrates and an HMG-COA reductase inhibitor is associated with an increased risk of a rare but serious skeletal muscle toxicity manifested by rhabdomyolysis, markedly elevated creatine phosphokinase (CPK) levels and myoglobinuria, acute renal failure, and sometimes death. The risk versus benefit of using this therapy should be determined for individual patients based on their risk of cardiovascular and/or pancreatic complications of hyperlipidemia.

The use of grapefruit, Seville oranges, and their products (juices, etc.) is not permitted from 1 week before enrollment and during treatment.

### Dose Modification

Tables below provide the guidance for dose modifications for CFG920 and Afuresertib.

TABLE 5A
Dose Modification Guidance for Thrombocytopenia in Phase I Cycle 1

| | | Dose Modification Suggestions | |
|---|---|---|---|
| Toxicity | Grade or Value | CFG920 | Afuresertib |
| Platelets | Grade 1 | Change BID daily dose to | None |
| | <LLN-75,000/mm³ | BID D1-5, Q7D (taking | |
| | | CFG920 75 mg from Monday | |
| | | to Friday and stop the | |
| | | Saturday and Sunday dose - | |
| | | taking 4 days per week) | |
| | | Resume BID daily dose | |
| | | again once Platelet count | |
| | | returns to baseline or >75,000/mm³ | |
| | Grade 2 | Hold treatment until toxicity | None |
| | <75,000-50,000/mm³ | resolves to baseline or >75,000/mm³ | |
| | Grade 3 | Hold treatment until toxicity | Hold treatment until toxicity |
| | <50,000-25,000/mm³ | resolves to baseline or >75,000/mm³ | resolves to grade 1 or less. |
| | Grade 4 | Withdraw patient from study. | Withdraw patient from study. |
| | <25,000/mm³ | | |

TABLE 5B
Dose Modification Guidance Phase I (Cycle 2 and beyond) and Phase II of the study

| | | Dose Modification Suggestions | |
|---|---|---|---|
| Toxicity | Grade or Value | CFG920 | Afuresertib |
| Platelets | Grade 1 | Change BID daily dose to | None |
| | <LLN-75,000/mm³ | BID D1-5, Q7D (taking | |
| | | CFG920 75 mg from | |
| | | Monday to Friday and stop | |
| | | the Saturday and Sunday | |
| | | dose - taking 4 days per | |
| | | week) | |
| | | Resume BID daily dose | |
| | | again once Platelet count | |
| | | returns to baseline or >75,000/mm³ | |
| | Grade 2 | Hold treatment until | None |
| | <75,000-50,000/mm³ | toxicity resolves to baseline | |
| | | or >75,000/mm³ | |
| | Grade 3 | Hold treatment until | Hold treatment until toxicity |
| | <50,000-25,000/mm³ | toxicity resolves to baseline | resolves to grade 1 or less. |
| | | or >75.000/mm³ | Reduce dose by 25 mg |
| | | Reduce dose by 25 mg | |
| | Grade 4 | Withdraw patient from | Withdraw patient from study. |
| | <25,000/mm³ | study. | |
| ANC | ≥500/mm³ to <1500/mm³ | None | None |
| | <500/mm³ | Hold treatment until ANC | Hold treatment until ANC |
| | | is ≥1500/mm³. Recommend | is ≥1500/mm³. Recommend |
| | | repeat ANC in 1 week if | repeat ANC in 1 week if |
| | | treatment held. Consider | treatment held. Consider |
| | | reduction of dose by 25 mg. | reduction of dose by 25 mg. |
| Febrile neutropenia | | If resolved <5 days, | If resolved <5 days, consider |
| (38.5° C. for 48 hours | | consider reducing dose by | reducing dose by 25 mg. |
| or longer) | | 25 mg. | |
| Peripheral sensory | Grade 1 | None | None |
| neuropathy | Grade 2 | None | None |
| | Grade 3 | None | None |
| | Grade 4 | Withdraw patient from | Withdraw patient from study. |
| | | study. | |
| Hyperglycemia | Grade 1-2 | None | None |
| | Grade 3 | Hold treatment. | Hold treatment. |
| | | If resolved to grade 1 | If resolved to grade 1 |
| | | <14 days, reduce dose | <14 days, reduce dose |
| | | by 25 mg. | by 25 mg. |
| | Grade 4 or ketoacidosis | Withdraw patient from | Withdraw patient from study. |
| | | study. | |
| Anaphylaxis (acute | Severe reactions defined as | None unless CFG920 is the | None unless afuresertib is the |
| hypersensitivity | hypotension requiring | only study drug being taken | only study drug being taken |
| reactions) | treatment, dyspnea | by the patient. In that case, | by the patient. In that case, |
| | requiring bronchodilators, | immediately discontinue | immediately discontinue |
| | angioedema or generalized | treatment and do NOT | treatment and do NOT |
| | urticaria. | rechallenge. | rechallenge. |
| Conduction disorder | Grade 1 and 2 | None | None |
| | Grade ≥3 | None | None |
| Other grade ≥3 non- | Grade ≥3 | Hold treatment. If resolved, | Hold treatment. If resolved to |
| hematological | | to baseline in <14 days, | baseline in <14 days, |
| toxicity except rash | | consider dose reduction by | consider dose reduction by |
| (refer to | | 25 mg. Consider re- | 25 mg. Consider re- |
| Section 6.4.1.2) | | escalation to full dose in | escalation to full dose in next |
| | | next cycle if no recurrence | cycle if no recurrence or new |
| | | or new toxicities. | toxicities. |

TABLE 6A
Schedule of Planned Study Assessments of Phase I Part

| | | | | | | | | | | | End of | Safety |
|---|---|---|---|---|---|---|---|---|---|---|---|---|
| | | | | | | | | | | | Treatment^{m} | Follow-Up |
| | | | | | | | | | | | within 15 | Within 30 |
| | | Cycle 1 | | | | | | Subsequent Cycles | | | days (±3 | days (±3 |
| | | | | | Day | Day | Day | | Day | | days) of the | days) of the |
| | Screening | | | | 8 | 15 | 22 | Day 1 | 15^{l} | Day | last dose of | last dose of |
| | Days (−28 | Day | Day | Day | (±1 | (±3 | (±3 | (±1 | (±3 | 28 (±7 | study | study |
| Procedure | to −1) | 1 | 2 | 4 | day) | days) | days) | day) | days) | days) | treatment | treatment |
| Informed consent^{a} | X | | | | | | | | | | | |
| Medical history^{b} | X | | | | | | | | | | | |
| Inclusion/exclusion | X | | | | | | | | | | | |
| criteria | | | | | | | | | | | | |
| Physical | X | Xⁿ | | | | | | Xⁿ | | | X | |
| examination | | | | | | | | | | | | |
| Vital signs^{c} | X | X^{r} | | X | X | X | X | X | X | | X | |
| ECOG | X | X | | | | | | X | | | X | |
| performance status | | | | | | | | | | | | |
| Height/Weight^{q} | X | | | | | | | X^{q} | | | | |
| 12-lead ECG^{d} | X | X^{r} | | X | | X | | | | | X | |
| Complete blood | X | X^{r} | | | X | X | X | X | X^{l} | | X | |
| count^{e} | | | | | | | | | | | | |
| Serum chemistry^{f} | X | X^{r} | | | X | X | X | X | X^{l} | | X | |
| PSA | X | | | | | | | X | | | X | |
| Serology (hepatitis | X | | | | | | | | | | | |
| B and C, HIV, | | | | | | | | | | | | |
| tuberculosis) | | | | | | | | | | | | |
| Urinalysis^{g} | X | | | | | | | | | | | |
| Pharmacokinetics^{h} | | X | X^{o} | | | X | | X^{o} | | | | |
| Pharmacodynamicsⁱ | | X^{o} | | | X^{o} | X^{o} | X^{o} | X^{o} | | | | |
| Tumor assessment^{j} | X | | | | | | | | | X^{p} | X^{p} | |
| Concomitant | X | X | X | X | X | X | X | X | X | X | X | X |
| medications | | | | | | | | | | | | |
| Administration of | | X | Continuous BID dosing | | | | | | | | | |
| CFG920 and | | | | | | | | | | | | |
| prednisone^{k} | | | | | | | | | | | | |
| Administration of | | X | Continuous QD dosing | | | | | | | | | |
| afuresertib^{k} | | | | | | | | | | | | |
| Dispense CFG920 | | | X | X | X | X | X | X | X | | | |
| and prednisone | | | | | | | | | | | | |
| Dispense | | | X | X | X | X | X | X | X | | | |
| afuresertib | | | | | | | | | | | | |
| Collect used and | | | | X | X | X | X | X | X | | X | |
| unused drug | | | | | | | | | | | | |
| containers and | | | | | | | | | | | | |
| perform | | | | | | | | | | | | |
| accountability | | | | | | | | | | | | |
| check | | | | | | | | | | | | |
| Antineoplastic | | | | | | | | | | | X | X |
| therapies since | | | | | | | | | | | | |
| discontinuation of | | | | | | | | | | | | |
| study treatment | | | | | | | | | | | | |
| PTEN IHC staining | X | | | | | | | | | | | |

| | | | |
|---|---|---|---|
| Adverse event | X | X | [symbol] |
| reporting | | | |

Abbreviations: ALT = alanine aminotransferase, AST = aspartate aminotransferase, BID = twice daily, CRPC = castration-resistant prostate cancer, CXDX = Cycle X Day X, CT = computed tomography, ECG = electrocardiogram, ECOG = Eastern Cooperative Oncology Group, EOT = end of treatment, HIV = human immunodeficiency virus, MRI = magnetic resonance imaging, pGSK3β = phosphorylated glycogen synthase kinase 3 beta, PSA = prostate-specific antigen, PTEN = phosphatase and tensin homolog, QD = once daily.
Note:
One Cycle = 28 days.
^{a}No study specific procedures should be performed prior to obtaining informed consent.
^{b}Medical history should be pertinent to the current study and include Gleason score at initial diagnosis, diagnosis and extent of cancer, prostate cancer history, concomitant illnesses and prior medications/treatments such as previous CRPC treatments, previous radiotherapy or past surgeries.
^{c}Vital signs (pulse, blood pressure, respiratory rate, temperature) will be collected during the study. Pulse and blood pressure will be obtained with the patient sitting and then after standing for 1 minute. After at least 2 minutes of rest, 2 blood pressure records should be measured with the patient sitting. Then the patient should stand for 1 minute and then 2 blood pressure measurements should be done in order to monitor for the development of hyper or hypotension.
^{d}All ECGs will be performed in duplicate, 5 minutes apart. If the trace is not normal, then a third one is required.
^{e}Complete blood count will consist of determinations of the white blood cell count, hemoglobin, white blood cell count (differential), and platelet count. Complete blood count will be assessed weekly during the assessment of dose-limiting toxicity and biweekly for Cycle 2, 3, 4, then once a month for the rest of subsequent Cycles, and the EOT visit.
^{f}Serum chemistry will consist of determinations of serum levels of sodium, potassium, chloride, bicarbonate, blood urea nitrogen, creatinine, glucose, ALT, AST, alkaline phosphatase, total bilirubin, direct bilirubin, calcium, magnesium, phosphorus, albumin, total protein, uric acid. Serum chemistry will be assessed weekly during the assessment of dose-limiting toxicity and biweekly for Cycle 2, 3, 4, then once a month for the rest of subsequent Cycles, and the EOT visit.
^{g}Urinalysis will consist of determinations of pH and specific gravity; and dipstick determinations of glucose, ketones, protein, bilirubin, and blood. If any of the dipstick determinations are 2+ or greater, a microscopic examination of the urine should also be performed.
^{h}Blood samples for measurement of CFG920 and afuresertib PK plasma concentrations will be collected on C1D1 and C1D15 (pre-dose, and 0.5, 1, 2, 4, 6, 8, 12, and 24 hours after the morning dose), and on Day 1 of subsequent Cycles (prior to morning dose).
Note:
The 12-hour samples on C1D15 should be collected before the next CFG920 dose on that day, and the 24-hour samples on C1D1 and C1D15 should be collected before the morning CFG920 and afuresertib doses on C1D2 and C1D16, respectively. Patients should be counseled that they should not take their morning doses on C1D2 and C1D16 until after they have visited the study unit and the PK samples have been collected.
The pre-dose sample on Day 1 should be collected within 1 hour before dosing, and the pre-dose sample on Day 15 should be collected within 10 minutes before morning dosing. For other time points, samples can be obtained ±2 minutes of the scheduled sampling time for samples to be collected ≤1 hour after a dose, ±5 minutes of the scheduled sampling time for samples to be collected >1 and ≤8 hours after a dose, and ±30 minutes of the scheduled sampling time for samples to be collected >8 and ≤24 hours after a dose.
ⁱPharmacodynamics analysis will consist of determinations of total testosterone, cortisol, aldosterone, adrenocorticotropic hormone, and plasma renin activity. Blood samples will be obtained within 1 hour prior to morning dose of CFG920 and afuresertib (pre-dose) on Days 1, 8, and 15 of Cycle 1, and Day 1 of Cycles 3, 8 and 24. The pre-dose sample on Day 1 should be collected within 1 hour before dosing, and the pre-dose sample on other days should be collected within 10 minutes before morning dosing.
^{j}Tumor assessment should include all pertinent imaging procedures to identify areas of metastatic disease, same method (eg, CT, MRI or bone scan) with same specification of tumor assessment should be used throughout the study.
^{k}Patients will be contacted on C1D13, to remind them to maintain an approximate 12-hour dosing interval for CFG920 and 24-hour dosing interval for afuresertib on C1D14 and C1D15, to maintain an adequate period of fasting around the times of CFG920 dosing, and to record the dosing dates and times for self-administered doses on C1D14. Patient's will also be reminded to report to the study unit at the correct time on C1D15, so that the pre-dose samples can be collected and the morning doses of CFG920 and afuresertib administered on time (i.e., approximately 12 hours after the evening dose of CFG920 on C1D14).
^{l}Day 15 visit and assessments in subsequent Cycle are only for Cycle 2, 3, 4, 6.
^{m}The EOT visit will occur 15 (±3) days after the last dose.
ⁿTargeted physical examination(s) focusing on areas involved by prostate cancer or adverse events (Digital rectal examination should be done at screening only).
^{o}Trough sample only (ie, prior to morning dose).
^{p}Tumor assessments to determine extent of disease will be obtained at the end (Day 28 ± 7 days) of Cycles 2, 4, 6; and every 3 treatment Cycles after Cycle 6, also obtained at the end of treatment, except the patient has completed tumor assessment within 28 days of EOT. Patients who end study treatment not due to progressive disease or death from any cause, should keep tumor assessment schedule after EOT if patients consent.
^{q}Height will be only collected at Screening in this study.
^{r}Procedures conducted during screening that are performed with 5 days of Day 1 can also be used as the Day 1 pre-dose evaluation and do not need to be repeated.

TABLE 6B
Schedule of Planned Study Assessments of Phase II Part

| | | | | | | | | | | | Overall |
|---|---|---|---|---|---|---|---|---|---|---|---|
| | | | | | | | | | | Safety | Survival |
| | | | | | | | | | EOT^{j} | Follow-Up | Follow- |
| | | | | | | | | | (±3 days) | Within 30 | Up |
| | | | | | | | | | Within 15 | days (±3 | Every 12 |
| | | Cycle 1 | | | | Subsequent Cycles | | | days (±3 | days) of | weeks |
| | | | | Day | Day | Day | Day | | days) of the | the last | (±7 |
| | Screening | | Day 8 | 15 | 22 | 1 | 15ⁱ | Day | last dose of | dose of | days) |
| | (Days −28 | | (±1 | (±3 | (±3 | (±1 | (±3 | 28 (±7 | study | study | after |
| Procedure | to −1) | Day 1 | day) | days) | days) | day) | days) | days) | treatment | treatment | EOT |
| Informed consent^{a} | X | | | | | | | | | | |
| Medical history^{b} | X | | | | | | | | | | |
| Inclusion/exclusion | X | | | | | | | | | | |
| criteria | | | | | | | | | | | |
| Physical | X | X^{k} | | | | X^{k} | | | X | | |
| examination | | | | | | | | | | | |
| Vital signs^{c} | X | Xⁿ | X | X | X | X | X | | X | | |
| ECOG | X | X | | | | X | | | | | |
| performance status | | | | | | | | | | | |
| Height/Weight^{m} | X | | | | | | | X^{m} | | | |
| 12-lead ECG^{d} | X | Xⁿ | | X | | | | | | | |
| Complete blood | X | Xⁿ | X | X | X | X | Xⁱ | | | | |
| count^{e} | | | | | | | | | | | |
| Serum chemistry^{f} | X | Xⁿ | X | X | X | X | Xⁱ | | | | |
| PSA | X | | | | | X | | | X | | |
| Serology (hepatitis | X | | | | | | | | | | |
| B and C, HIV, | | | | | | | | | | | |
| tuberculosis) | | | | | | | | | | | |
| Urinalysis^{g} | X | | | | | | | | | | |
| Tumor assessment^{h} | X | | | | | | | X^{l} | X^{l} | | |
| PTEN IHC | X | | | | | | | | | | |
| staining | | | | | | | | | | | |
| Concomitant | X | X | X | X | X | X | X | X | X | X | |
| medications | | | | | | | | | | | |
| Administration of | | X | Continuous BID dosing | | | | | | | | |
| CFG920 and | | | | | | | | | | | |
| prednisone | | | | | | | | | | | |
| Administration of | | X | Continuous QD dosing | | | | | | | | |
| afuresertib | | | | | | | | | | | |
| Dispense CFG920 | | X | X | X | X | X | X | | | | |
| and prednisone | | | | | | | | | | | |
| Dispense | | X | X | X | X | X | X | | | | |
| afuresertib | | | | | | | | | | | |
| Collect used and | | | X | X | X | X | X | | X | | |
| unused drug | | | | | | | | | | | |
| containers and | | | | | | | | | | | |
| perform | | | | | | | | | | | |
| accountability | | | | | | | | | | | |
| check | | | | | | | | | | | |
| Antineoplastic | | | | | | | | | X | X | |
| therapies since | | | | | | | | | | | |
| discontinuation of | | | | | | | | | | | |
| study treatment | | | | | | | | | | | |
| Survival Contact | | | | | | | | | | | X |

| | |
|---|---|
| Adverse event | [symbol] |
| reporting | |

Abbreviations: ALT = alanine aminotransferase, AST = aspartate aminotransferase, BID = twice daily, CRPC = castration-resistant prostate cancer, CXDX = Cycle X Day X, CT = computed tomography, ECG = electrocardiogram, ECOG = Eastern Cooperative Oncology Group, EOT = end of treatment, HIV = human immunodeficiency virus, MRI = magnetic resonance imaging, PSA = prostate-specific antigen, PTEN = phosphatase and tensin homolog, QD = once daily.
Note:
One Cycle = 28 days.
^{a}No study specific procedures should be performed prior to obtaining informed consent.
^{b}Medical history should be pertinent to the current study and include Gleason score at initial diagnosis, diagnosis and extent of cancer, prostate cancer history, concomitant illnesses and prior medications/treatments such as previous CRPC treatments, previous radiotherapy or past surgeries.
^{c}Vital signs (pulse, blood pressure, respiratory rate, temperature) will be collected during the study. Pulse and blood pressure will be obtained with the patient sitting and then after standing for 1 minute. After at least 2 minutes of rest, 2 blood pressure records should be measured with the patient sitting. Then the patient should stand for 1 minute and then 2 blood pressure measurements should be done in order to monitor for the development of hyper or hypotension.
^{d}All ECGs will be performed in duplicate, 5 minutes apart. If the trace is not normal, then a third one is required.
^{e}Complete blood count will consist of determinations of the white blood cell count, hemoglobin, white blood cell count differential and platelet count. Complete blood count will be assessed weekly during the assessment of dose-limiting toxicity and biweekly for Cycle 2, 3, 4, then once a month for the rest of subsequent Cycles.
^{f}Serum chemistry will consist of determinations of serum levels of sodium, potassium, chloride, bicarbonate, blood urea nitrogen, creatinine, glucose, ALT, AST, alkaline phosphatase, total bilirubin, direct bilirubin, calcium, magnesium, phosphorus, albumin, total protein, uric acid. Serum chemistry will be assessed weekly during the assessment of dose-limiting toxicity and biweekly for Cycle 2, 3, 4, then once a month for the rest of subsequent Cycles.
^{g}Urinalysis will consist of determinations of pH and specific gravity; and dipstick determinations of glucose, ketones, protein, bilirubin, and blood. If any of the dipstick determinations are 2+ or greater, a microscopic examination of the urine should also be performed.
^{h}Tumor assessment should include all pertinent imaging procedures to identify areas of metastatic disease, same method (eg, CT, MRI or bone scan) with same specification of tumor assessment should be used throughout the study.
ⁱDay 15 visit and assessments in subsequent Cycle are only for Cycle 2, 3, 4.
^{j}The EOT visit will occur 15 (±3) days after the last dose.
^{k}Targeted physical examination(s) focusing on areas involved by prostate cancer or adverse events (Digital rectal examination should be done at screening only).
^{l}Tumor assessments to determine extent of disease will be obtained at the end (Day 28 ± 7 days) of Cycles 2, 4, 6; and every 3 treatment Cycles after Cycle 6, also obtained at the end of treatment, except the patient has completed tumor assessment within 28 days of EOT. Patients who end study treatment not due to progressive disease or death from any cause, should keep tumor assessment schedule after EOT if patients consent.
^{m}Height will be only collected at Screening in this study.
ⁿProcedures conducted during screening that are performed within 5 days of Day 1 can also be used as the Day 1 pre-dose evaluation and do not need to be repeated.

### Example C: Summary of the Efficacy and Safety of the Combination Therapy With CFG920+Afuresertib in a Human Patient

One of the objectives of the study in Example B was to evaluate a patient who received a combination therapy dose of CFG920 75 mg+prednisone 5 mg BID+Afuresertib 100 mg QD in Cohort 1. This patient demonstrated anti-cancer efficacy as assessed by Prostate Specific Antigen (PSA) after receiving the combination therapy of CFG920+prednisone+AFURESERTIB treatment. This patient's clinical manifestation has been summarized below to support the patent application.

Medical History: This study involved a 79-year-old Caucasian man with a past medical history of Atrial fibrillation, hypertension, acid reflux, insomnia, and glaucoma; and was treated with Digoxin, Lisinopril/Metoprolol, aluminum hydroxide/simethicone, Temazepam, Timolol/Travoprost, respectively, for each of the above disorders. He was diagnosed with prostate cancer at the age of 66 years old. He received radical prostatectomy shortly after his prostate cancer was diagnosed, although he received Androgen-Deprivation Therapy (ADT), his prostate cancer progressed to metastasis castration-resistant prostate cancer (mCRPC) including bone metastasis 8 years later. Thereafter, this patient received multiple lines of anti-cancer treatments, such as Abiraterone, Cabazitaxel, Enzalutamide, radium 223 (Xofigo), docetaxel (Taxotere) and Denosumab (Xgeva), but his tumor was still progressed before enrollment in the study described in Example B.

Study Treatment: After he met all the inclusion and exclusion criteria, this patient was enrolled in the Phase I stage of this study as a patient in Cohort 1 to receive the starting combination therapy dose of CFG920 75 mg+prednisone 5 mg BID+Afuresertib 100 mg QD. Unfortunately, this patient received a higher Afuresertib dose of CFG920 75 mg+prednisone 5 mg BID+Afuresertib 150 mg QD from Day 57 to 85 (Cycle 3), a total of 28 days, due to an operation error. The error was corrected and the dose was reverted to CFG920 75 mg+prednisone 5 mg BID+Afuresertib 100 mg QD in Day 1 of Cycle 4 (after Day 85) as summarized below:
Cycle 1 (Day 1 to 28): CFG920 75 mg BID+Afuresertib 100 mg QD
Cycle 2 (Day 29 to 56): CFG920 75 mg BID+Afuresertib 100 mg QD
Cycle 3 (Day 57 to 85): CFG920 75 mg BID+Afuresertib 150 mg QD
Cycle 4 (Day 86 to 114): CFG920 75 mg BID+Afuresertib 100 mg QD
Cycle 5 (Day 115 to 118): CFG920 75 mg BID+Afuresertib 100 mg QD (Cut-off date is Jul. 23, 2020, Day 118)

Anti-Cancer Efficacy of the Combination Therapy: PSA is a major surrogate marker to measure the progress of prostate cancer (Scott Williams. Surrogate endpoints in early prostate cancer research. Transl Androl Urol. 2018 June; 7(3): 472-482). During the study, the PSA response was defined as a decline of ≥50% from baseline, according to the Prostate Cancer Working Group 3 (PCWG3) criteria (Howard I. Scher, Michael J. Morris, Walter Michael Stadler, Celestia S. Higano, Susan Halabi, Matthew Raymond Smith et al., The Prostate Cancer Working Group 3 (PCWG3) consensus for trials in castration-resistant prostate cancer (CRPC). Journal of Clinical Oncology, 2015, Volume 33, Issue 15_suppl).

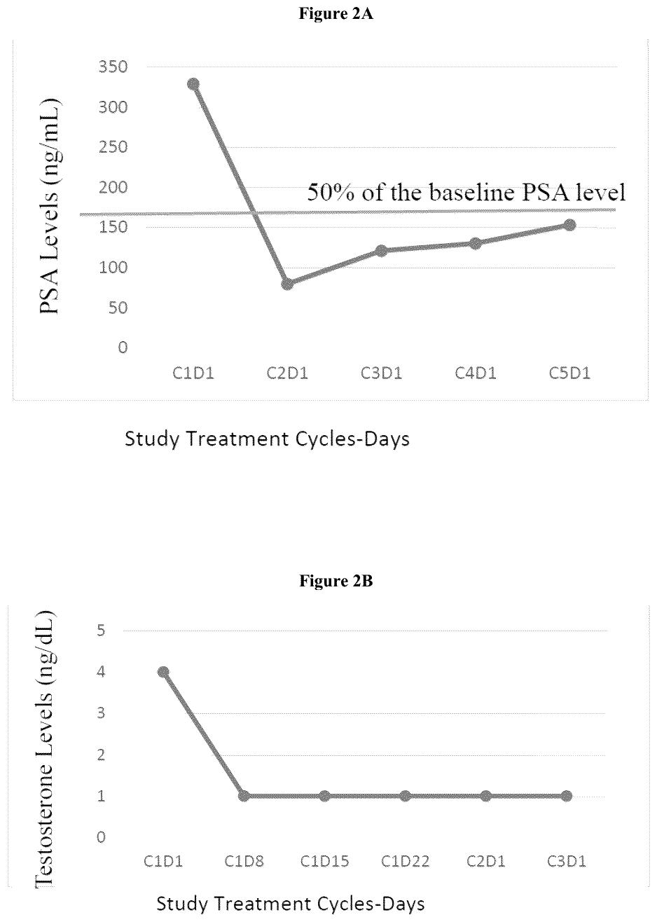

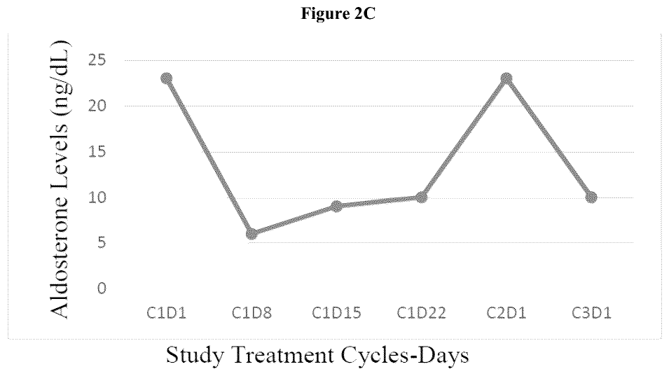

The PSA level of the patient was reduced more than 50% from the baseline PSA level from Dayl of Cycle 2 (i.e., Day 29) to Day 1 of Cycle 5 (i.e., Day 115) with the cut-off date of Jul. 23, 2020 as shown in FIG. 2A. The anti-cancer efficacy of the combination therapy shown in FIG. 2A was achieved mainly by the dose of CFG920 75 mg+Prednisone 5 mg BID+Afuresertib 100 mg QD because the higher combined dose of CFG920 75 mg+Prednisone 5 mg BID+Afuresertib 150 mg QD treatment started only on Day 1 of Cycle 3 (i.e., Day 57), which was 4 weeks later from the initial PSA decline date Day 1 of Cycle 2 (i.e., Day 29). During the Cycle 3, Day 1 to Cycle 3, Day 28, although treated by a higher combination dose of CFG920 75 mg+prednisone 5 mg BID+Afuresertib 150 mg QD, patient's PSA levels did not reduce further, and rather slightly increased, as compared with the PSA levels in Cycle 1 and Cycle 2.

Another critical tumor progression assessment is the tumor image study based on PCWG3, which uses the bone scan, PSA response and Response Evaluation Criteria in Solid Tumors (RECIST) 1.1 in prostate cancer (Lawrence H. Schwartz, Lesley Seymour, Saskia Litière, et al. RECIST 1.1-Standardisation and disease-specific adaptations: Perspectives from the RECIST Working Group. Eur J Cancer. 2016 July; 62:138-145). In this study, the tumor image assessment based on RECIST 1.1 is the primary endpoint of this study. This patient's tumor image assessments were reported as a stable disease condition during the study treatment based on RECIST 1.1 criteria. The patient demonstrated solid PSA responses (<50% of baseline level) and a stable condition of his prostate cancer was assessed by PCWG3 criteria for more than 112 days under the treatment of the combination therapy with CFG920+prednisone+Afuresertib.

Pharmacodynamic Marker: Androgenic hormones are widely accepted to regulate proliferation, apoptosis, angiogenesis, metastasis, and differentiation of prostate cancer (Takashi Imamoto, Hiroyoshi Suzuki, Masashi Yano, Koji Kawamura, Naoto Kamiya, Kazuhiro Araki, Akira Komiya, Naoki Nihei, Yukio Naya and Tomohiko Ichikawa. The role of testosterone in the pathogenesis of prostate cancer. International Journal of Urology (2008) 15, 472-480). Testosterone, one of the major androgen hormones, is a widely used pharmacodynamic marker of the anti-androgen treatment for prostate cancer in many studies. The changes of testosterone level under the study treatment are shown in FIG. 2B. The results demonstrated that the combination therapy with CFG920+prednisone+Afuresertib could effectively inhibit the testosterone production in this patient. This pharmacodynamic marker provided further evidence of the effectiveness of the combination anti-cancer therapy of CFG920 75 mg+prednisone 5 mg BID+Afuresertib 100 mg QD, although it is only the starting doses in this study, the results show that this combination and dose could effectively reduce the levels of both PSA and androgen in this patient.

CFG920 inhibits both CYP11B and CYP17A1: CFG920 is a dual enzyme inhibitor for both CYP11B and CYP17A1 that not only inhibits androgen production with anti-cancer activities, but also inhibits aldosterone synthesis to prevent hyperaldosteronism. Hyperaldosteronism is a disorder with several serious clinical symptoms including hypertension, hypokalemia, fatigue, headache, muscle weakness and numbness. Results from FIG. 2B demonstrated that from the baseline (before study treatment) to Day 1 of Cycle 3, the aldosterone level in different time points are lower than the baseline level except for Day 1 of Cycle 2, which is a similar aldosterone level as in the baseline. In addition, this patient did not report any adverse event (AE) like the symptoms of hyperaldosteronism (see Table 7). The results of FIG. 2B, FIG. 2C and Table 7 provide solid evidence that CFG920 has the dual enzyme inhibition and did not cause any clinical symptoms of hyperaldosteronism during the treatment with the combination therapy.

Safety of Combination Therapy: this patient reported 4 AEs with grade 1/mild in severity rating as shown in the Table 7 below. Although the patient received a higher dose of Afuresertib (150 mg QD) during Cycle 3, after carefully assessing the days of AEs and the dose of study treatments, there is no correlation between the time of AEs occurred and study treatment doses because all 4 AEs occurred in the Cycle 1, whereas the higher dose of Afuresertib of 150 mg QD was taken in Cycle 3. There is more than 1 month gap between the AEs and the patient's taking of the higher dose of Afuresertib. In addition, no AE was reported in Cycle 3, although this is the Cycle that the patient received the higher dose of Afuresertib 150 mg QD. Furthermore, all 4 reported AEs are benign in nature and mild in severity without any significant clinical consequence related to the combination therapy in this study.

TABLE 7
List of the Adverse Events (AES) of Patient

| | | AE START | AE START | | AE | AND | Body System or | RELATED TO STUDY |
|---|---|---|---|---|---|---|---|---|
| AE ID | AE TERM | DATE | CYCLE-DAY | AE END DATE | OUTCOME | SEVRITY | Organ Class | TREATMENT? |
| 1 | Polyuria | Apr. 5, 2020 | C1D4 | NA | NOT YET | Grade 1: | Renal and | VERY LIKELY |
| | | | | | RECOVERED | Mild | urinary | |
| | | | | | | | disorders | |
| 2 | Nausea | Apr. 13, 2020 | C1D12 | NA | NOT YET | Grade 1: | Gastrointestinal | VERY LIKELY |
| | | | | | RECOVERED | Mild | disorders | |
| 3 | Thrombo | Apr. 13, 2020 | C1D12 | Apr. 14, 2020 | RECOVERED | Grade 1: | Blood and | VERY LIKELY |
| | cytopenia | | | 0:00 | | Mild | lymphatic | |
| | | | | | | | system | |
| | | | | | | | disorders | |
| 4 | Dysgeusia | Apr. 26, 2020 | C1D24 | NA | NOT YET | Grade 1: | Nervous system | VERY LIKELY |
| | | | | | RECOVERED | Mild | disorders | |

Various modifications of the invention, in addition to those described herein, will be apparent to those skilled in the art from the foregoing description. Such modifications are also intended to fall within the scope of the appended claims. Each reference, including all patent, patent applications, and publications, cited in the present application is incorporated herein by reference in its entirety.

1. A method of treating castrate resistant prostate cancer in a patient, comprising:
determining that the patient is resistant to one or more treatments comprising one or more of abiraterone, enzalutamide, apalutamide, darolutamide, and cabazitaxel, or a pharmaceutically acceptable salt thereof, or a combination thereof; and
administering to the patient:
(i) N-{(1S)-2-amino-1-[(3-fluorophenyl)methyl]ethyl}-5-chloro-4-(4-chloro-1-methyl-1H-pyrazol-5-yl)-2-thiophenecarboxamide (afuresertib), or a pharmaceutically acceptable salt thereof; and
(ii) 1-(2-chloro-pyridin-4-yl)-3-(4-methyl-pyridin-3-yl)-imidazolidin-2-one (CFG920), or a pharmaceutically acceptable salt thereof;
wherein afuresertib, or a pharmaceutically acceptable salt thereof, is administered to the patient in a total daily dosage of from about 75 mg to about 150 mg, on a free base basis; and
wherein CFG920, or a pharmaceutically acceptable salt thereof, is administered to the patient twice per day (BID) in a total daily dosage of about 100 mg or about 150 mg, on a free base basis.

2-3. (canceled)

4. The method of claim 1, wherein afuresertib is in the form of a crystalline hydrochloride salt.

5. The method of claim 1, wherein CFG920 is in the form of the anhydrous free base.

6. (canceled)

7. The method of claim 1, wherein afuresertib, or a pharmaceutically acceptable salt thereof, is administered to the patient in a total daily dosage of from about 75 mg to about 100 mg, on a free base basis.

8. The method of claim 1, wherein afuresertib, or a pharmaceutically acceptable salt thereof, is administered to the patient in a total daily dosage of about 75 mg, about 100 mg, about 125 mg or about 150 mg, on a free base basis.

9. The method of claim 1, wherein afuresertib, or a pharmaceutically acceptable salt thereof, is administered to the patient once daily (QD).

10-11. (canceled)

12. The method of claim 1, wherein CFG920, or a pharmaceutically acceptable salt thereof, is administered to the patient in a dosage of about 50 mg or about 75 mg, twice per day (BID).

13. (canceled)

14. The method of claim 1, wherein the patient is administered a twice daily dosage of about 50 mg per dose of CFG920, or a pharmaceutically acceptable salt thereof, on a free base basis; and a once daily dosage of about 75 mg per dose of afuresertib, or a pharmaceutically acceptable salt thereof, on a free base basis.

15. The method of claim 1, wherein the patient is administered a twice daily dosage of about 50 mg per dose of CFG920, or a pharmaceutically acceptable salt thereof, on a free base basis; and a once daily dosage of about 100 mg per dose of afuresertib, or a pharmaceutically acceptable salt thereof, on a free base basis.

16. The method of claim 1, wherein the patient is administered a twice daily dosage of about 50 mg per dose of CFG920, or a pharmaceutically acceptable salt thereof, on a free base basis; and a once daily dosage of about 125 mg per dose of afuresertib, or a pharmaceutically acceptable salt thereof, on a free base basis.

17. The method of claim 1, wherein the patient is administered a twice daily dosage of about 75 mg per dose of CFG920, or a pharmaceutically acceptable salt thereof, on a free base basis; and a once daily dosage of about 75 mg per dose of afuresertib, or a pharmaceutically acceptable salt thereof, on a free base basis.

18. The method of claim 1, wherein the patient is administered a twice daily dosage of about 75 mg per dose of CFG920, or a pharmaceutically acceptable salt thereof, on a free base basis; and a once daily dosage of about 100 mg per dose of afuresertib, or a pharmaceutically acceptable salt thereof, on a free base basis.

19. The method of claim 1, wherein the patient is administered a twice daily dosage of about 75 mg per dose of CFG920, or a pharmaceutically acceptable salt thereof, on a free base basis; and a once daily dosage of about 125 mg per dose of afuresertib, or a pharmaceutically acceptable salt thereof, on a free base basis.

20-22. (canceled)

23. The method of claim 1, further comprising administering to the patient is a corticosteroid.

24. The method of claim 23, wherein the corticosteroid is prednisone.

25. The method of claim 24, wherein the prednisone is administered to the patient in a total daily dosage of about 10 mg, BID.

26. The method of claim 24, wherein the patient is administered a twice daily dosage of about 50 mg per dose of CFG920, or a pharmaceutically acceptable salt thereof, on a free base basis; a twice daily dosage of about 5 mg per dose of prednisone; and a once daily dosage of about 75 mg per dose of afuresertib, or a pharmaceutically acceptable salt thereof, on a free base basis.

27. The method of claim 24, wherein the patient is administered a twice daily dosage of about 50 mg per dose of CFG920, or a pharmaceutically acceptable salt thereof, on a free base basis; a twice daily dosage of about 5 mg per dose of prednisone; and a once daily dosage of about 100 mg per dose of afuresertib, or a pharmaceutically acceptable salt thereof, on a free base basis.

28. The method of claim 24, wherein the patient is administered a twice daily dosage of about 50 mg per dose of CFG920, or a pharmaceutically acceptable salt thereof, on a free base basis; a twice daily dosage of about 5 mg per dose of prednisone; and a once daily dosage of about 125 mg per dose of afuresertib, or a pharmaceutically acceptable salt thereof, on a free base basis.

29. The method of claim 24, wherein the patient is administered a twice daily dosage of about 75 mg per dose of CFG920, or a pharmaceutically acceptable salt thereof, on a free base basis; a twice daily dosage of about 5 mg per dose of prednisone; and a once daily
dosage of about 75 mg per dose of afuresertib, or a pharmaceutically acceptable salt thereof, on a free base basis.

30. The method of claim 24, wherein the patient is administered a twice daily dosage of about 75 mg per dose of CFG920, or a pharmaceutically acceptable salt thereof, on a free base basis; a twice daily dosage of about 5 mg per dose of prednisone; and a once daily dosage of about 100 mg per dose of afuresertib, or a pharmaceutically acceptable salt thereof, on a free base basis.

31. The method of claim 24, wherein the patient is administered a twice daily dosage of about 75 mg per dose of CFG920, or a pharmaceutically acceptable salt thereof, on a free base basis; a twice daily dosage of about 5 mg per dose of prednisone; and a once daily dosage of about 125 mg per dose of afuresertib, or a pharmaceutically acceptable salt thereof, on a free base basis.

32-34. (canceled)

35. The method of claim 24, wherein the corticosteroid and CFG920, or a pharmaceutically acceptable salt thereof, are administered simultaneously.

36. The method of claim 24, wherein the corticosteroid and CFG920, or a pharmaceutically acceptable salt thereof, are co-formulated.

37. The method of claim 1, wherein afuresertib, or a pharmaceutically acceptable salt thereof, and CFG920, or a pharmaceutically acceptable salt thereof, are administered simultaneously.

38. The method of claim 1, wherein afuresertib, or a pharmaceutically acceptable salt thereof, and CFG920, or a pharmaceutically acceptable salt thereof, are administered sequentially.

39. The method of claim 1, wherein:
(i) afuresertib, or a pharmaceutically acceptable salt thereof, is administered once per day and
(ii) CFG920, or a pharmaceutically acceptable salt thereof, is administered twice per day, wherein afuresertib, or a pharmaceutically acceptable salt thereof, is administered simultaneously with one of the two daily dosages of CFG920, or a pharmaceutically acceptable salt thereof.

40. The method of claim 1, wherein afuresertib, or a pharmaceutically acceptable salt thereof, is formulated as part of a pharmaceutically acceptable composition further comprising one or more pharmaceutically acceptable excipients.

41. The method of claim 1, wherein CFG920, or a pharmaceutically acceptable salt thereof, is formulated as part of a pharmaceutically acceptable composition further comprising one or more pharmaceutically acceptable excipients.

42. The method of claim 1, wherein afuresertib, or a pharmaceutically acceptable salt thereof, is administered to the patient orally.

43. The method of claim 1, wherein CFG920, or a pharmaceutically acceptable salt thereof, is administered to the patient orally.

44. The method of claim 1, wherein the patient is additionally administered an androgen deprivation therapy.

45. The method of claim 44, wherein the androgen deprivation therapy is a luteinizing hormone releasing hormone agonist or antagonist.

46. The method of claim 44, wherein the androgen deprivation therapy is sufficient to maintain castration levels of serum testosterone in the patient.

47. The method of claim 1, wherein the castration resistant prostate cancer is metastatic castration resistant prostate cancer.
